(19) United States
(12) Patent Application Publication
(10) Pub. No.: US 2022/0027816 A1
(43) Pub. Date: Jan. 27, 2022

(54) WORKPLACE DISRUPTION DATA AND PROTOCOLS

(71) Applicant: Cox Automotive, Inc., Altanta, GA (US)

(72) Inventors: Duane Ritter, Atlanta, GA (US); Chris Kirk, Atlanta, GA (US); Jim Shortal, Atlanta, GA (US); Jessie Lacks, Atlanta, GA (US); Jonathan Smoke, Atlanta, GA (US); Mark Strand, Atlanta, GA (US); Taylor Horton, Atlanta, GA (US); Russell Anderson, Atlanta, GA (US); Spencer Taft, Atlanta, GA (US); Russ Johnson, Atlanta, GA (US); Viktor Wronowski, Atlanta, GA (US)

(73) Assignee: Cox Automotive, Inc., Atlanta, GA (US)

(21) Appl. No.: 17/382,248

(22) Filed: Jul. 21, 2021

Related U.S. Application Data
(60) Provisional application No. 63/054,690, filed on Jul. 21, 2020.

(51) Int. Cl. G06Q 10/06; G06N 7/00; G16H 50/80

(52) U.S. Cl. CPC G06Q 10/06312; G06Q 10/0633; G16H 50/80; G06N 7/005; G06Q 10/063116

(57) ABSTRACT

This disclosure describes systems, methods, and devices related to customizing the presentation of data indicative of disruptive events relative to workplaces. A device may determine a geographical area comprising one or more geographical sub-areas, and a disruptive event is taking place at the geographical area. The device may determine one or more metrics associated with the disruptive event. The device may determine, based on the one or more metrics, a first disruption score indicative of a future severity condition caused by the disruptive event that will take place at a first geographical sub-area of the one or more geographical sub-areas. The device may generate a first user interface to present a geographical map associated with the geographical area, and the first geographical area is marked by the first disruption score.

### CROSS-REFERENCE TO RELATED APPLICATION(S)

This application claims the benefit of U.S. Provisional Application No. 63/054,690, filed Jul. 21, 2020, the disclosure of which is incorporated herein by reference as if set forth in full.

### TECHNICAL FIELD

This disclosure generally relates to systems, methods, and devices for the generation and presentation of workplace disruption data.

### BACKGROUND

The frequency of natural and manmade disasters may cause workplace disruptions of varying severity at different locations. Examples of natural disasters include hurricanes, earthquakes, and pandemics. Examples of manmade disasters include drastic changes in economic conditions and geopolitical tensions leading to widespread labor unrest and war. Such disaster events may be disruptive to a workplace. However, data indicating what has happened or is currently happening may not facilitate future workplace protocol decisions.

### BRIEF DESCRIPTION OF THE DRAWINGS

FIGS. 1-13 depict illustrative schematic diagrams for an example of a workplace disruption dashboard, in accordance with one or more example embodiments of the present disclosure.

FIG. 14 depicts a table of an example of work disruption levels, in accordance with one or more example embodiments of the present disclosure.

FIG. 15 depicts a table of example work disruption protocols, in accordance with one or more example embodiments of the present disclosure.

FIG. 16 depicts example tables of work disruption levels and corresponding procedural guidelines, in accordance with one or more example embodiments of the present disclosure.

FIG. 17 depicts an example table of work disruption levels and corresponding guidelines for workplace visitors and contractors, in accordance with one or more example embodiments of the present disclosure.

FIG. 18 depicts example tables of travel restrictions, in accordance with one or more example embodiments of the present disclosure.

FIG. 19 depicts a flow diagram of an illustrative process for generating a work disruption dashboard, in accordance with one or more embodiments of the disclosure.

FIG. 20 depicts a diagram illustrating an example network environment for generating a workplace disruption dashboard, in accordance with one or more example embodiments of the present disclosure.

FIG. 21 depicts a block diagram of an example machine upon which any of one or more techniques (e.g., methods) may be performed, in accordance with one or more example embodiments of the present disclosure.

### DETAILED DESCRIPTION

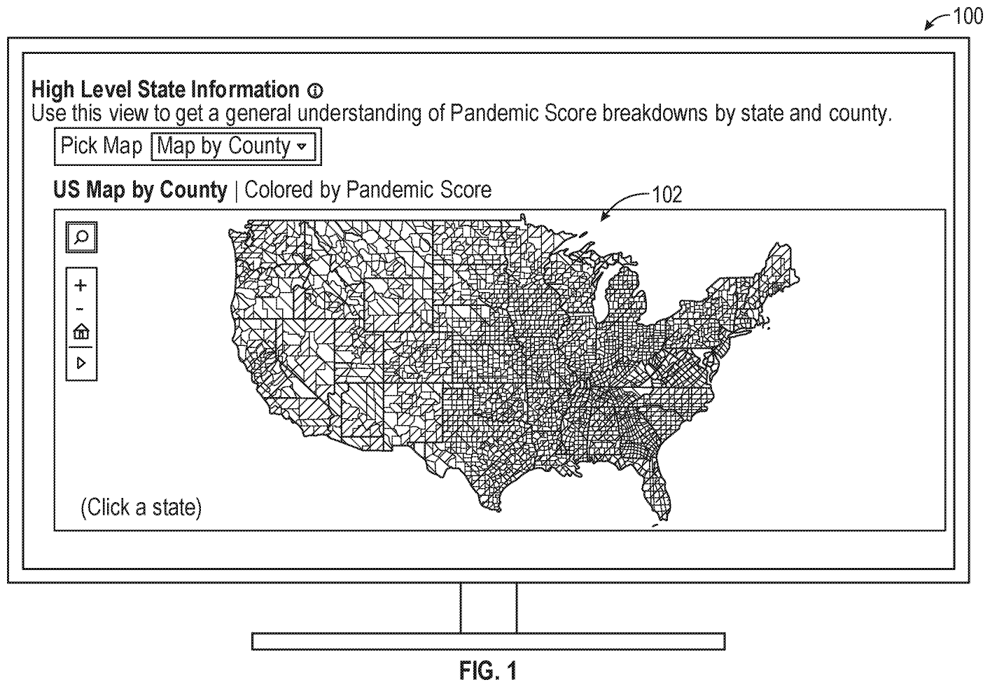

Example embodiments described herein provide certain systems, methods, and devices for customized analysis and presentation of data indicative of disruptive events relative to workplaces. These systems, methods, and devices may particularly provide improved user interfaces (which may also be referred to herein as “dashboard(s)”) for viewing and interacting with real-time data relating to a disruptive event and its impact (for example, impact on operation of a business). Examples of such user interfaces may be illustrated below in FIGS. 1-13.

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

Data-driven decision making is playing an important role in determining how businesses and other organizations make workplace decisions, such as whether to open or close physical locations, the number and type of employees who are permitted to be present at a given workplace location, whether customers may visit the premises of a workplace location, whether employees and/or customers need to wear personal protective equipment (PPE) while at a workplace location, whether travel restrictions should be in place, and the like. In particular, organizations with workplace locations in multiple geographic areas (e.g., cities, states, countries, etc.) may experience different disruptions at different workplace locations at any time, thereby resulting in difficult decisions by organization stakeholders regarding how to respond to and/or prepare for conditions of a workplace disruption (e.g., the inability of employees to physically be at a workplace, travel restrictions, etc.). Not only is aggregation and analysis of data such as people affected by a virus or weather event time consuming, but yesterday's or today's data may not allow a stakeholder to make a decision regarding whether a location may need to close, may be allowed to reopen, and to what extent, in the future.

In addition, organizations may implement a variety of protocols that allow and/or restrict behavior of employees and/or customers during disruptive events (e.g., natural and manmade disasters, disruption caused by bad weather, and/or rally, or the like). Such protocols may not be the only governing protocols. For example, governments may implement additional restrictions or impose additional requirements on organizations. Whether a stakeholder may implement a policy governing operation of a workplace at a given location may depend not only on past and/or current event data, but on future predictions and the alignment of organizational protocols with government laws and regulations. With such data changing in real-time, the ability of a stakeholder to identify the right information to analyze to make decisions about current and future working conditions may be limited. Also, computer-based systems that rely on organizational protocols may not be aware of present or projected protocol changes, and the implementation of such changes may not occur instantaneously.

Therefore, people and computer systems may benefit from customized analysis and presentations of workplace disruption data that project future changes in workplace protocol.

Illustrative embodiments of the systems and methods described herein may generally be directed to, among other things, determining and presenting various workplace disruption levels for different workplace locations. A workplace disruption level may be indicative of the severity and an amount of disruption caused by a disruptive event, and which protocols (e.g., defining allowed and restricted actions) may be implemented. For example, one disruption level may indicate that employees may not physically be at a workplace. Another disruption level may indicate that employees physically may be at a workplace, but with some restrictions (e.g., personal protective equipment, social distancing, etc.). The workplace disruption level may be defined by one or more disruption scores as further described below. A disruptive event may be an event disrupting a workplace. Examples of a disruptive event may include natural disasters (e.g., hurricanes, earthquakes, pandemics, and the like), manmade disasters (e.g., drastic changes in economic conditions and geopolitical tensions leading to widespread labor unrest and war, or the like), an event caused by bad weather, an event caused by large crowds, or any other events that may cause a workplace disruption. The presentation of workplace disruption levels and underlying data may facilitate return-to-work and/or workplace closure/restriction decisions based on medical, governmental and geographical data-driven triggers presented using a workplace disruption dashboard. The present disclosure may not only help organizations decide if and when to return or send home employees or customers, but also to project future increases and decreases in workplace restrictions, thereby allowing stakeholders to prepare for and/or implement protocols before a situation changes. For example, workplace restrictions during a pandemic may be minimal given current data that indicates a relatively low workplace disruption level, but when the disruption data indicates that the workplace disruption level may be more significant within a matter of days, rather than waiting for the disruption data to confirm that more restrictive workplace protocols need to be implemented, stakeholders may begin preparations for changing workplace protocols before being required to implement them.

The systems, methods, and devices may also employ predictive algorithms that may be used to analyze real-time data and forecast future metrics associated with the disruptive events. For example, if the disruptive event is a pandemic-type event, the predictive algorithms may be used to determine a herd immunity date for various regions. The predicted herd immunity date may also be automatically adjusted in real-time based on changes in input data. For example, the predicted herd immunity date may be based on a number of vaccine doses being administered, and depending on how the number of administered doses changes on a day-to-day basis, the herd immunity date may be automatically adjusted. The predictive algorithms may also be used to forecast any other types of information, which may depend on the type of disruptive event that is being tracked using the systems, methods, and devices described herein. This forecasted information may also be presented to a user through the user interfaces in a number of different forms (for example, text, plots, maps, etc.).

According to example embodiments of the present disclosure, a computer system may generate a workplace disruption dashboard to present a map that indicates workplace disruption scores at a geographical scale (e.g., at a county scale, at a zip code scale, at a state scale, a city scale, at an area code scale, and/or at any other scale associated with a boundary area). A computer system may identify geographic areas, determine respective workplace disruption scores for the geographic areas, and may present the geographic areas with their respective disruption scores (e.g., using a color-coded map whose colors indicate the disruption scores). The computer system may use one or more graphical user interfaces to present the geographic areas and their respective disruption scores. When a user of the computer system selects a geographic area (e.g., by hovering over the area, clicking the area, touching the area, etc.), the computer system may present one or more additional graphical user interfaces concurrently or in place of the map interface to present a customized display of data relevant to the disruption score, including a projected change in the score (e.g., the score will increase or decrease in a number of days). Examples are described in FIGS. 1-13.

According to example embodiments of the present disclosure, a workplace disruption score may be indicative of a disruptive event that is taking place and/or will take place at a particular geographical location and/or area, the effects (e.g., health, travel, etc.) of the disruptive event at the particular geographical location/area, and which protocols (e.g., defining allowed and restricted actions) may be implemented at the particular geographical location/area (or to travel to/from that location). For instance, a workplace disruption score may be indicative of the current level and/or a future level of disruption or threat of disruption caused by a disruptive event, such as the severity of a virus or illness (e.g., the number of people who contract the virus or illness, the morbidity rate of a virus or illness, etc.), the severity of a weather disruption, the number of road or other transportation closures caused by the disruptive event, and the like. Based on the workplace disruption score, a workplace protocol may be implemented. A workplace protocol may define restrictions to be applied, which area and/or buildings may be closed or opened (and at what capacity), whether or not international or domestic travel may be permitted, the number of people allowed to be at a particular location, whether customers may be at a workplace premise, whether employees may visit customers (e.g., customer service calls), and the like.

In some embodiments, a computer system may determine a workplace disruption score associated with a geographical location based on medical, governmental and geographical data-driven triggers including but not limited to data associated with effects of an illness or virus, government rules, data associated with health system capacity and testing availability, data associated with personal protective equipment (PPE) and other key supplies, economic data, data associated with consumer sentiment at the geographical location, public data (e.g., school closings and public transit), vaccine data, hospitalization rates and hospital capacity levels, and/or any other data that may affect the score determination. In some embodiments, the computer system may determine a workplace disruption score based on one or more metrics associated with a geographical location. Examples of metrics may include a number of total cases associated with the geographical location/area, a change in the number of total cases compared with a prior time of period (e.g., one or more prior days, and/or one or more prior weeks), a number or percentage of total deaths caused by an event/illness/pandemic associated with the geographical location/area, a change in the number of total death cases compared with a prior time of period, and/or a number of newly added cases associated with the geographical location/area, a change in the number of newly added cases compared with a prior time of period. The computer system may determine a trend of the effects of a disruptive event (e.g., number of people who contract a virus) over time (e.g., daily, weekly, and/or with a predefined time interval). For example, the computer system may compare the score with one or more threshold scores indicating whether the score is in a particular score (e.g., severity) range (e.g., not severe, mildly severe, moderately severe, extremely severe, etc.). For instance, if a score falls within a particular score range, a computer system may present the score and/or range/level of the geographical location. In some embodiments, the computer system may determine a workplace disruption score based on a prediction model (e.g., a machine learning model). For example, the computer system may train a machine learning model using historic metrics such that the machine leaning model may learn how likely metrics falls at a particular severity level. The computer system may utilize the trained machine learning model to estimate a workplace disruption score and determine a corresponding severity level/range in which the score falls. In some embodiments, the above metrics used to determine the score may be weighted. For example, metrics associated with deaths may be weighted more heavily than metrics associated with newly added cases.

According to example embodiments of the present disclosure, a workplace disruption level may be defined by a workplace disruption score range indicative of a particular severity range (e.g., not severe, mildly severe, moderately severe, extremely severe, etc.). For instance, if a workplace disruption score falls within a particular workplace disruption level, a computer system may determine that a disruptive event is taking place or will take place at a particular severity range. The computer system may further determine one or more protocols that define which actions may be taken at a geographical location/area and recommend corresponding actions.

The computer system may not only provide a workplace disruption level (also referred as to a projected workplace disruption level) indicative of a future level of disruption or threat of disruption caused by a disruptive event, but also provide timing information indicating that when the future level will occur. In addition, the computer system may determine a protocol change based on a change of the workplace disruption level (e.g., a change from a workplace disruption level indicative of a mildly severe condition to a projected workplace disruption level indicative of an extremely severe condition, or the like). The computer system may allow for stakeholders to take actions to allow for the implementation of the protocols associated with the projected workplace disruption level so that workplace environments are able to implement the protocols immediately upon the protocol change that corresponds with the workplace disruption level change. By providing a customized display of relevant workplace disruption scores and/or levels, such as concurrent projected score/level presentations with relevant protocol data, stakeholders quickly may identify preparations to begin implementation of protocols governing workplace environments. In some embodiments, the computer system may generate a map marked by various colors indicative of workplace disruption scores/levels at a geographical scale, and may overlay the geographic interface with underlying data, score/level projections, and/or protocol data, to allow stakeholders to navigate maps with multiple locations and quickly identify protocols to implement based on projected workplace disruption scores/levels and associated data (e.g., historical and/or current data used to determine projected workplace disruption scores, and/or projected data associated with a future level of disruption or threat of disruption caused by a disruptive event).

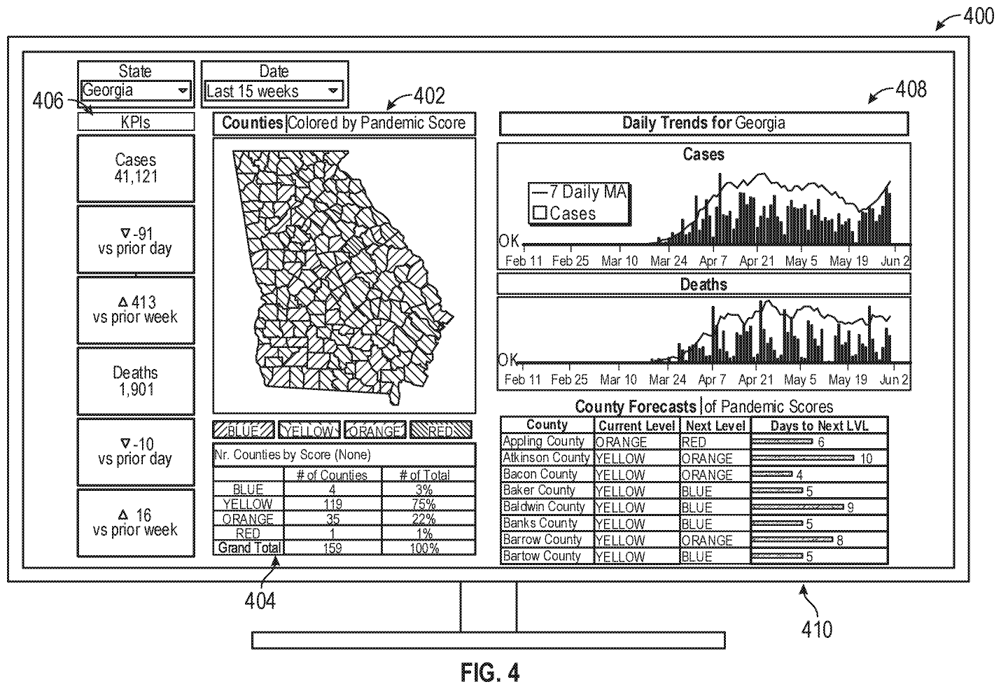

The computer system may present information associated with a geographical location/area that is selectable by a user. For instance, a user may click on a geographical location/area (e.g., the state of Georgia). The computer system may generate a user interface with which to display the selected geographical location/area, and may separately or concurrently present the user interface with or in place of a map that presents workplace disruption scores/levels for other locations. For instance, the computer system may present the state-level user interface in a separate window than a country-level or region-level map, or may present the state-level user interface at least partially overlaying the generated map. The state-level user interface may present state-level locations such as a counties within the state, zip codes within the state, cities within the state, and/or at any other scale associated with a boundary area. The state-level interface may indicate workplace disruption scores/levels by county, city, area code, etc. In some embodiments, the computer system may also generate and present real time and/or historical metrics associated with the event at the geographical scale. Example of metrics may include a number of total cases associated with the geographical location/area, a change in the number of total cases compared with a prior time of period (e.g., one or more prior days, and/or one or more prior weeks), a number of total death cases associated with the geographical location/area, a change in the number of total death cases compared with a prior time of period, and/or a number of newly added cases associated with the geographical location/area, a change in the number of newly added cases compared with a prior time of period. The computer system may determine a trend of the effects of a disruptive event (e.g., number of people who contract a virus) over time (e.g., daily, weekly, and/or with a predefined time interval). Additionally and/or alternatively, the computer system may generate a prediction model based on the above metrics to predict a future trend indicative of when (e.g., a number of days) a projected workplace disruption score/level may be achieved by the geographic area (e.g., a change from a moderate to a severe score/level, or vice versa). For instance, a county may be at a less severe level (e.g., most of employees may be permitted to work at a workplace). The computer system may determine that that county may reach a more severe level (e.g., most of employees may be required to work from home) after a time period (e.g., in a couple of days, weeks, and/or month). An example is further described in FIG. 4.

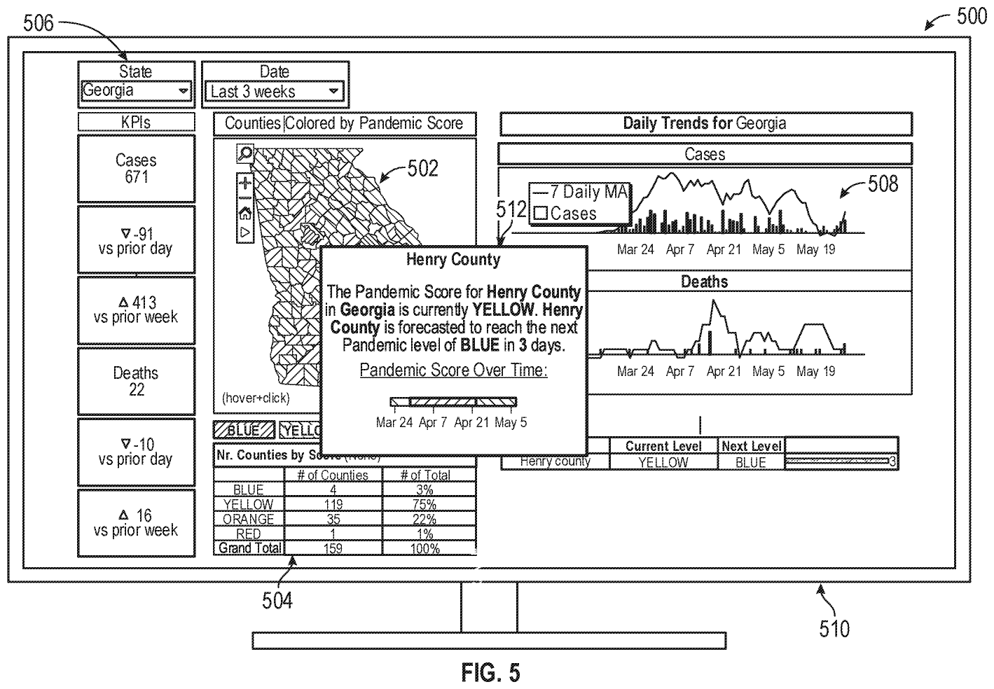

In some embodiments, the computer system may generate and present a user interface for a particular sub-area (e.g., a county, a city and/or a boundary area) within a selected geographical location/area. For instance, the computer system may present the user interface in a separate window or present the user interface at least partially overlaying the state-level interface. The user interface may include metrics associated with the particular sub-area, e.g., a current workplace disruption score/level, a projected workplace disruption score/level and after what time period from the current time (e.g., the number of days from now that the workplace disruption score may change), the past, current and future workplace disruption scores/levels are over a predefined time period, the number of locations with respective workplace disruption scores/levels, the number of people impacted by a disruption event, and the like. The above metrics may be presented in text, a plot, or any other format relevant to presenting the metrics. In some embodiments, when the particular sub-area is selected on the sub-map, the computer system may filter out metrics that are associated with other sub-areas on the sub-map, and may present metrics associated with the particular sub-area. An example is further described in FIG. 5.

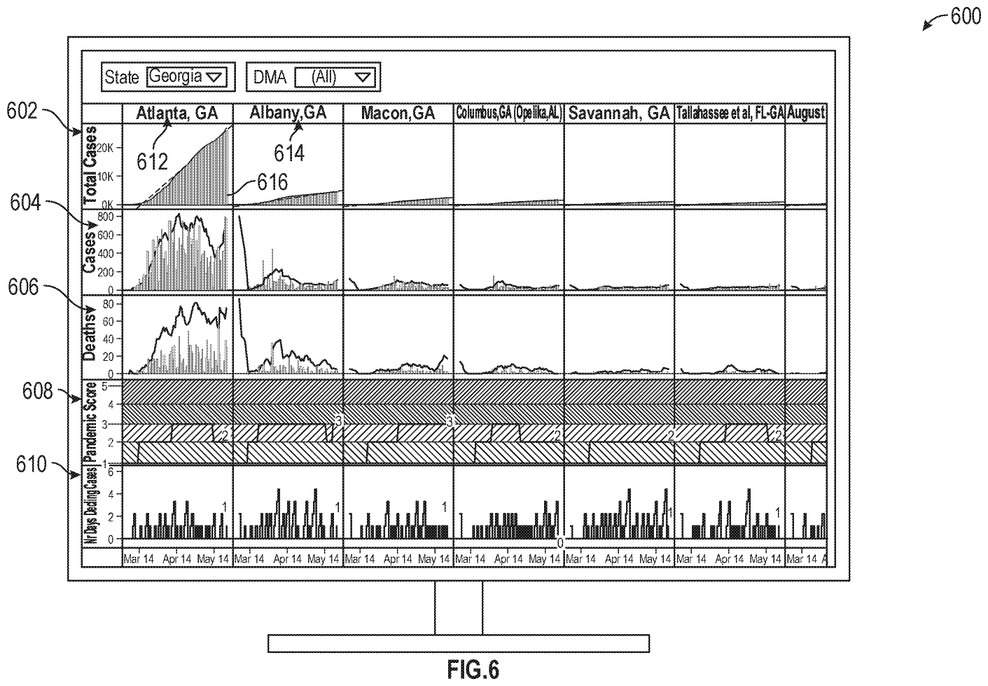

In some embodiments, the computer system may generate and present metrics for different areas (e.g., a county, a city and/or a boundary area) within the selected geographical location/area. The computer system may determine a comparison of metrics associated with different areas. For instance, the computer system may plot any metric over a predefined time period for any geographic area. Examples of metrics may include a number of total cases (e.g., a number of infected/ill people) associated with a disruptive event in a particular area, a number of newly added cases (e.g., a number of infected/ill people) associated with the disruptive event in the particular area, a number of total death cases associated with the disruptive event in the particular area, severity ranges associated with the particular area, a number of declining cases associated, previous and future weather patterns, actual and projected event crowd sizes, and/or any metric indicative of a workplace disruption in a particular area. An example is further described in FIG. 6. As such, the computer system provides a comprehensive analysis for users to better understand a situation where a disruptive event is taking place and/or will take place, and to make a decision about how to return to work, which areas, facilities and/or buildings may be reopened/restored, where and/or when to lift restrictions of protocols, and how to improve safety and security for traveling to a destination place and/or returning back to a workplace.

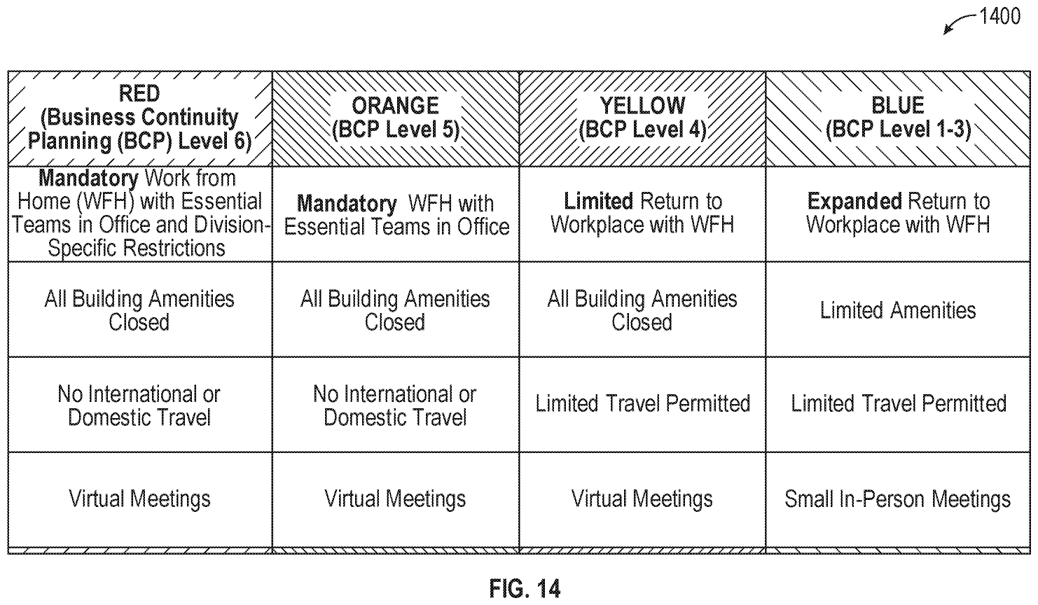

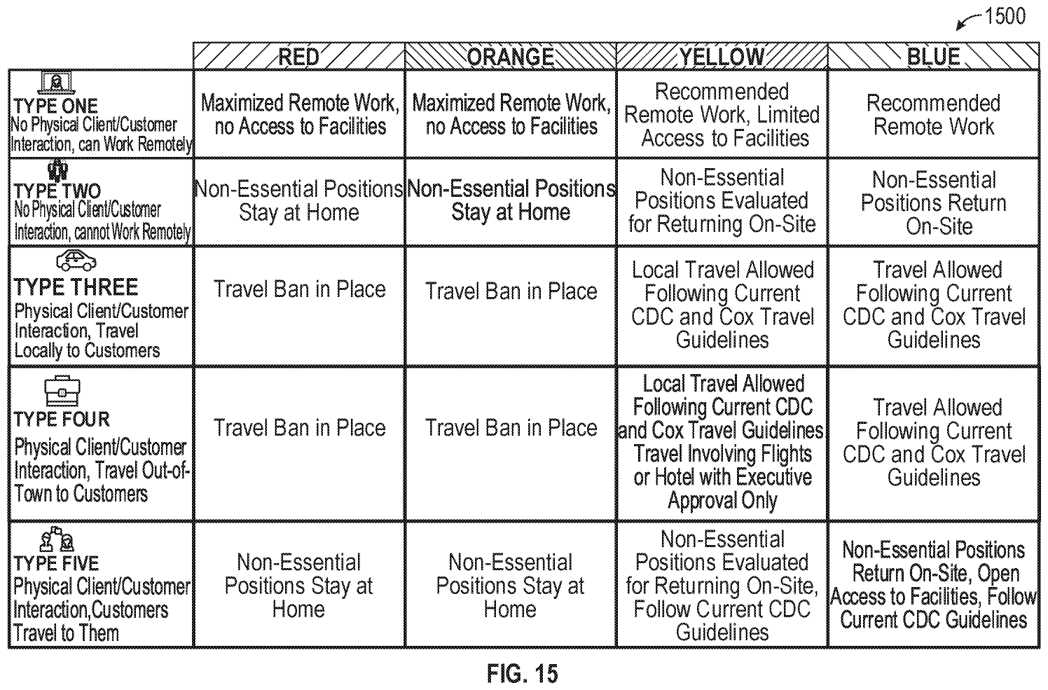

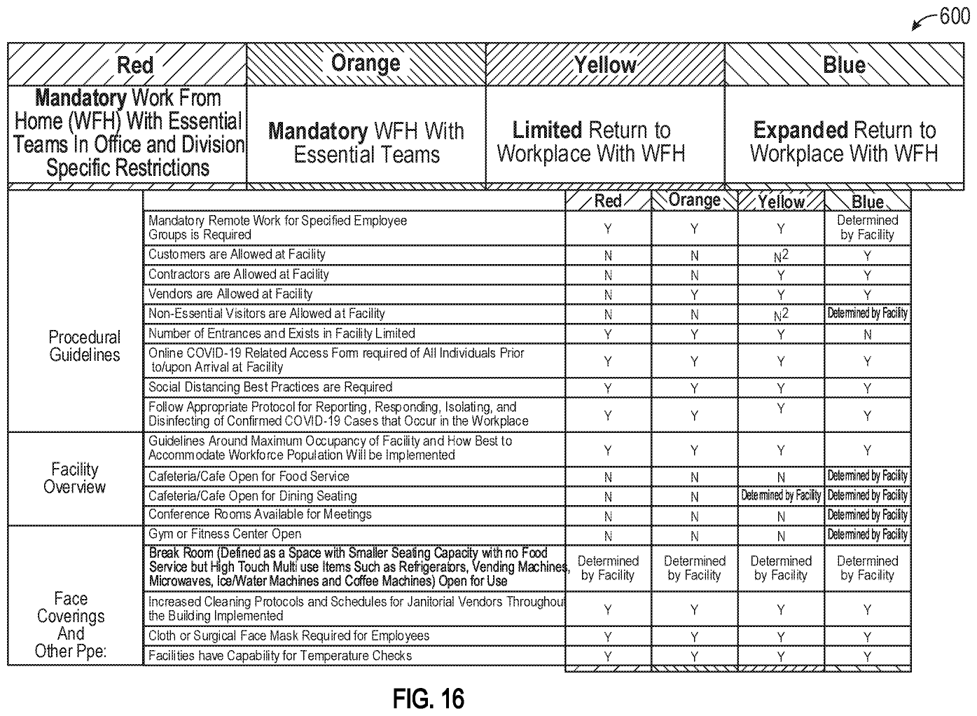

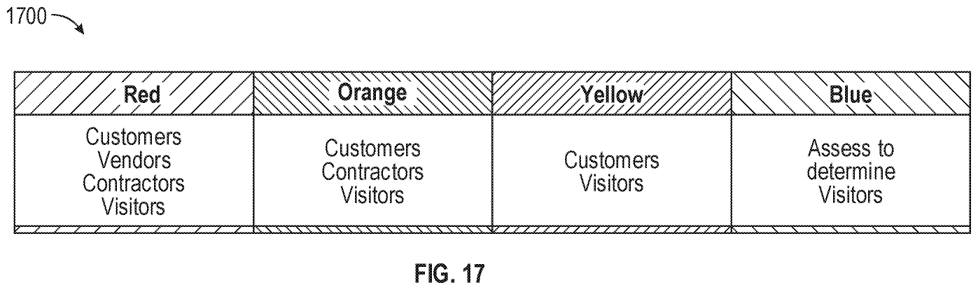

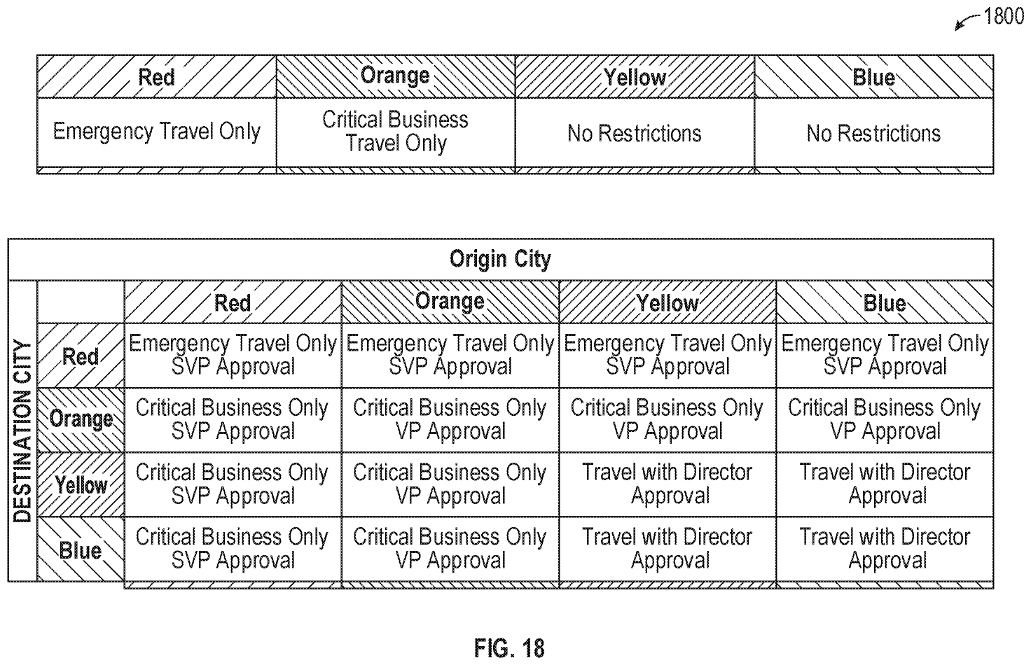

According to example embodiments of the present disclosure, a computer system may determine whether or not a user may work from home (WFH) at a geographical location based on workplace disruption scores/levels. For instance, a computer system may identify workplace protocols based on workplace disruption scores/levels as described above. In some embodiments, a protocol for a particular workplace disruption score/level may govern how many and/or what types of employees who may work at the workplace or may WFH, which buildings may be closed, whether or not international or domestic travel may be permitted and may require particular levels of supervisory permission, and/or how many people may be allowed to gather. Examples are described in FIG. 14. In some embodiments, a computer system may determine workplace disruption levels for various categories of employees or other stakeholders. For instance, the computer system may determine various categories of employees based on job functions and travel requirements, and the computer system may determine levels whose protocols govern whether or not employees of any category may WFH, stay at home, and/or travel locally or non-locally (e.g., outside of a metropolitan area). Examples are described in FIG. 15. In some embodiments, a computer system may determine workplace disruption levels indicative of which procedural guidelines may be applied, which facilities may be closed, and which PPE may be required to be worn, etc. Examples are described in FIG. 16. In some embodiments, a computer system may determine workplace disruption levels indicative of which types of people (e.g., customers, vendors, contractors, and/or visitors) may be allowed to access a particular facility, and/or particular areas of a facility (e.g., some physical areas may be more restricted than others). Examples are described in FIG. 17. In some embodiments, a computer system may determine workplace disruptive levels indicative of which types of travel (e.g., emergency travel and/or critical business travel) approved by which level of managers (e.g., senior vice president, vice president, and/or director) may be permitted at a particular departure geographical location and/or a destination location. Examples are described in FIG. 18.

Additionally, in some embodiments, the systems, methods, and devices described herein may involve performing any number of actions based on workplace disruption scores/levels. For example, if a given workplace disruption score crosses a threshold, then any of such actions may be taken. Examples of actions may include automatically providing notification on any of the dashboards described herein, automatically sending a notification to one or more users, or automatically enacting a particular policy. For example, if the workplace disruption scores/levels are associated with one or more operating regions of a place of business, employees of the place of business may automatically be provided notifications (for example, through mobile devices or otherwise) when the workplace disruption scores/levels cross a particular threshold. The place of business may also automatically be closed and certain systems associated with the place of business may be shut down when the thresholds are crossed. The actions may include any other number of suitable actions as well.

The above descriptions are for purposes of illustration and are not meant to be limiting. Numerous other examples, configurations, processes, etc., may exist, some of which are described in detail below. Example embodiments will now be described with reference to the accompanying figures.

FIGS. 1-13 depict illustrative schematic diagrams for an example of a workplace disruption dashboard, in accordance with one or more example embodiments of the present disclosure. For consistency sake, FIGS. 1-13 may illustrate a specific example use case where a workplace disruption includes a pandemic event. However, as mentioned above, a workplace disruption may include any other type of event (weather disasters, for example), and the use case presented in FIGS. 1-13 may simply be exemplary. FIG. 1 shows a workplace disruption dashboard 100 with which to present a map 102 (for example, a United States map or a map of any country), and the colors/shades of the map 102 may indicate workplace disruption scores/levels at a county scale. Multiple workplace disruption score ranges (also referred as to workplace disruption levels) may be marked by a first color/shade indicative of a county having an extremely severe disruption and which protocols may be implemented, a second color/shade indicative of a county having a moderately severe disruption, a third color/shade indicative of a county having a mildly severe disruption, and a fourth color/shade indicative of a county having a not-severe disruption. A user may select a particular scale (e.g., a zip code scale, a state scale, a city scale, an area code scale, and/or any other scale associated with a boundary area) at the top of the workplace disruption dashboard 100. The user may select a particular state (e.g., the state of Nebraska) at the bottom of the workplace disruption dashboard 100 to see state-level data associated with a workplace disruption. It should be noted that while the dashboards illustrated herein (for example, any of the dashboards illustrated in FIGS. 1-13 or otherwise) depict specific regions of the world, the dashboard may similarly present information for any other region of the world as well. For example, the dashboard 100 may not necessarily be limited to the United States, but may also present information for Canada, China, or any other region at any scale (for example, continents, countries, states, counties, provinces, etc.).

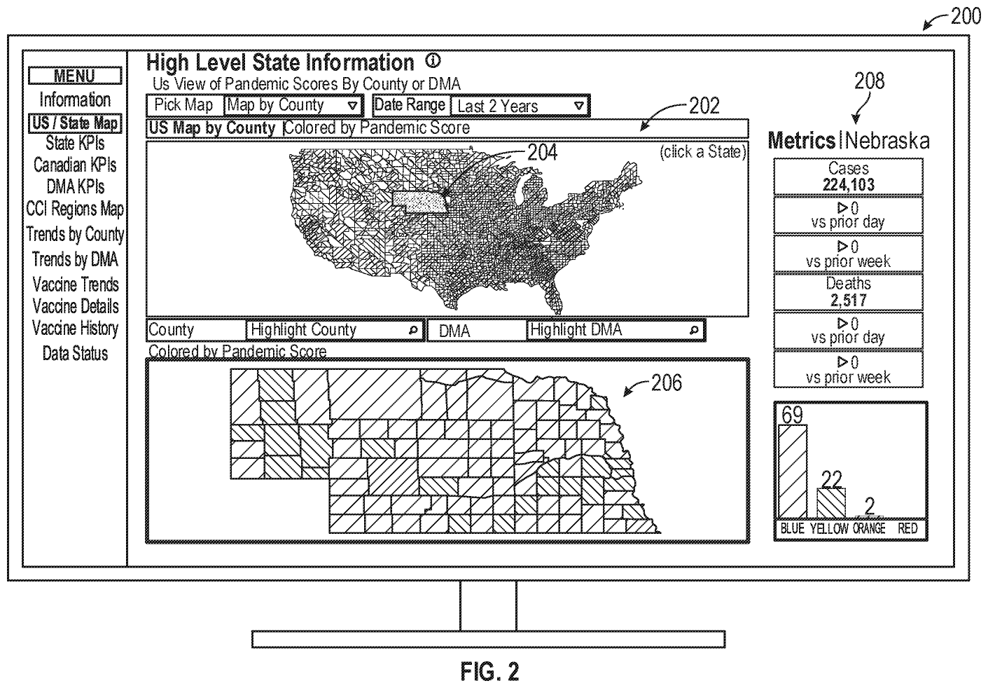

FIG. 2 shows a workplace disruption dashboard 200 presenting a map 202 of a larger region, and a second map 206 presenting a zoomed-in map of a sub-region of the larger region that may be selected by a user. For example, if the map 202 is a map of the United States as illustrated in the figure, a user may select a sub-region of the map 202, such as selectable portion 204. Upon selection, a second map 206 may be presented that displays a zoomed-in view of the sub-region that is selected by the user. As an example, the figure may depict the selectable portion 204 as including the state of Nebraska, such that the second map 206 may include a map of Nebraska. The second map 206 may thus depict the state of Nebraska and its counties, the individual counties having workplace disruption scores indicated by coloring/shading. The map may be presented by a user selecting the state of Nebraska at the bottom of the workplace disruption dashboard 200 in FIG. 2. This second map 206 may allow a user to better view visual information for the sub-region that is presented in the map 302. Additionally, the dashboard 200 may present one or more metrics 208. In the example provided in the figure, the metrics may include a total number of reported ases of a disease being tracked, a change in the number of reported cases, a number of deaths associated with the disease, and a change in the number of deaths. These metrics 208 may automatically adjust when the user selects a sub-region of the map 202. For example, when the user selects the sub-region 204 associated with Nebraska, the metrics 208 may be adjusted such that they include metrics associated only with Nebraska, rather than metrics associated with the United States as a whole.

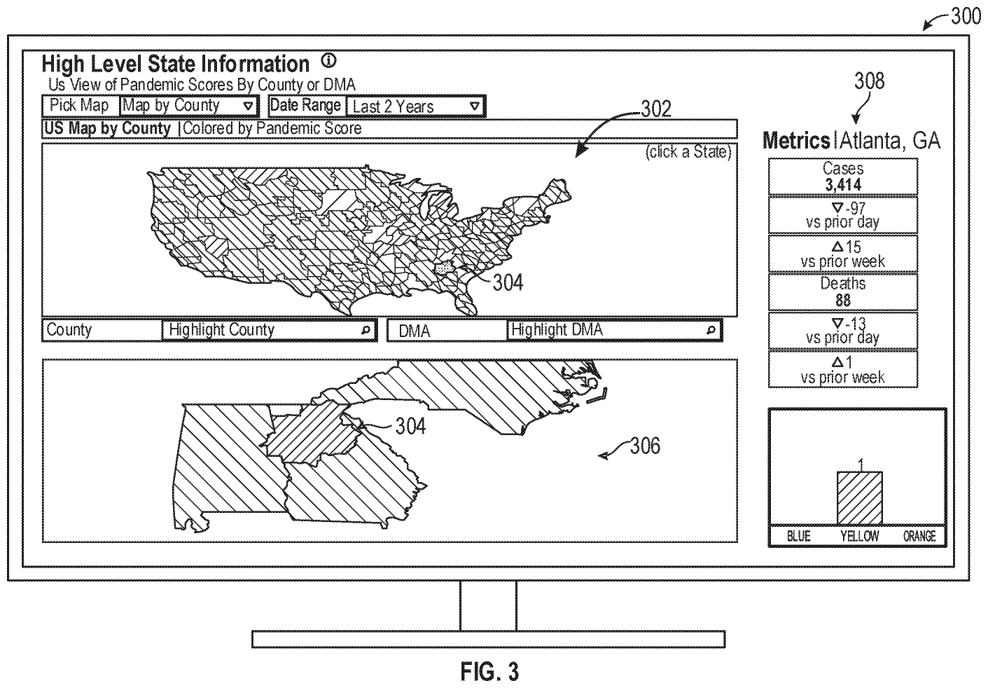

FIG. 3 also shows a workplace disruption dashboard 300 presenting a map 302 of a larger region, and a second map 306 that shows a zoomed-in map 306 of a sub-region 304 that may be selected by a user. The dashboard 300 may be the same as, or similar to, the dashboard 200 presented in FIG. 2. That is, the map 302 may be the same as, or similar to, the map 202, the metrics 308 may be the same as, or similar to, the metrics 308, etc. The dashboard 300 may differ from the dashboard 200 in that it may illustrate that the user may not necessarily be limited to selecting a particular state (if the map 302 is a map of the United States) or other type of pre-established geographical boundary, but rather the dashboard 300 may allow the user to select another sub-region that may include a portion a single state or may include a portion that encompasses multiple states. For example, the second map 306 may depict a sub-region 304 including portions of Georgia, Alabama, and North Carolina. The user may also be able to manually draw a boundary to indicate a sub-region of the map 302, where the sub-region included within the drawn boundary may be presented as the second map 306. The user may also be able to manually select the region in any other manner as well. As the sub-region presented in the second map 306. Additionally, since the sub-region 304 may include multiple states, the metrics 308 presented in the dashboard 300 may be metrics associated with the individual sub-region 304, rather than the one or more states that encompass the sub-region 304.

FIG. 4 shows a workplace disruption dashboard 400 presenting a map of the state of Georgia and its counties, the individual counties having workplace disruption scores indicated by coloring/shading. The map may be presented by a user selecting the state of Georgia from a map of any of the workplace disruption dashboards described herein (for example, the map 102, the map 202, the map 302, or any other map). The workplace disruption score ranges of FIG. 1 may be used at the county level of a selected state (e.g., Georgia). A user may select a “Date” range for which the workplace disruption data is to be presented (e.g., the last 15 weeks), and as a result, the dashboard may present the state map and associated metrics 406 corresponding to the selected date and the selected state, either in an interface that is displayed concurrently with the state-level map, or in an interface that is presented instead of the state-level map (not shown). The metrics 406 associated with the state (e.g., a number of total virus cases when the disruptive event is a viral pandemic, a change in the number of total virus cases compared with a prior day and a prior week, a number of total death cases due to a virus, a change in the number of total death cases a prior day and a prior week) are shown on the right of the map, and may be presented in any number of interfaces at least partially concurrently, separately, and/or using overlapping interfaces. For each colored pandemic score range, the number of counties and a percentage of the total number of the counties are shown below the map. On the left of the map, a plot 408 of the number of total cases (e.g., a number of infected/ill people) associated with the state for each day versus a time period and a plot of the number of death cases associated with the state for each versus the same time period show a trend of the effects of the pandemic. A table 410 of county forecasts may show a current workplace disruption level, a projected workplace disruption level and timing information when a county will reach to the projected workplace disruption level for a county list that may be selected by a user. The state-level data may be presented based on a viewer's subscription, viewer preferences, and the like, which may govern which information is shown, where, how respective interfaces presenting different data may be presented concurrently, and the like. For example, some of the state-level data may be presented at least partially concurrently with the state and/or country map, and/or with country-level data.

FIG. 5 shows a workplace disruption dashboard 500 to present information associated with a selected county when a user hovers over the county 501, clicks the county 501, and/or touches the county 501. A user interface 512 hovering over the state map 502 shows a current pandemic score range, a projected pandemic score range, and pandemic scores over a predetermined time period in text. That is, the dashboard 500 may illustrate that some or all of the elements presented in any of the dashboards described herein may be intractable. That is, elements within a given dashboard may be configured such that a user may hover over the element, select the element, or otherwise interact with the element in order to receive more details information with the element (such as the additional information associated with the specific county 501 as depicted in the figure). The metrics 506 associated with the county (e.g., a number of total virus cases, a change in the number of total cases compared with a prior day and a prior week, a number of total death cases, a change in the number of total death cases a prior day and a prior week) are shown on the right of the map. For each colored pandemic score range, the number of counties and a percentage of the total number of the counties are shown below the map. On the right of the map, a plot 508 of the number of total cases (e.g., a number of infected/ill people) associated with the county for each day versus a time period and a plot of the number of death cases associated with the county for each versus the same time period show a trend of the effects of the pandemic. A table 510 of county forecasts shows a current workplace disruption level, a projected workplace disruption level and timing information when the county will reach to the projected workplace disruption level for a county list that may be selected by a user. Information associated with non-selected counties are filtered out in the workplace disruption dashboard 500. The county-level data may be presented based on a viewer's subscription, viewer preferences, and the like, which may govern which information is shown, where, how respective interfaces presenting different data may be presented concurrently, and the like. For example, some of the county-level data may be presented at least partially concurrently with the state-level map and/or data.

FIG. 6 shows a workplace disruption dashboard 600 to present a comparison of metrics associated with different counties of the state of Georgia. Examples of metrics may include a number of total cases (e.g., a number of infected/ill people) for a particular county, a number of newly added cases (e.g., a number of infected/ill people) for the particular county, a number of total death cases for the particular county, pandemic scores associated with the particular county, a number of declining cases associated. The number of days of declining or increasing cases may be used to determine whether a trend is occurring, and whether to project a change of a workplace disruption score. For example, one day of declining or increasing cases may not indicate a trend, but three or more consecutive days of declining or increasing cases may indicate a trend that may be used to project a workplace disruption score increase or decrease within a number of days. Although the dashboard 600 presents metrics associated with counties in the state of Georgia, metrics may similarly be presented for any other geographical regions.

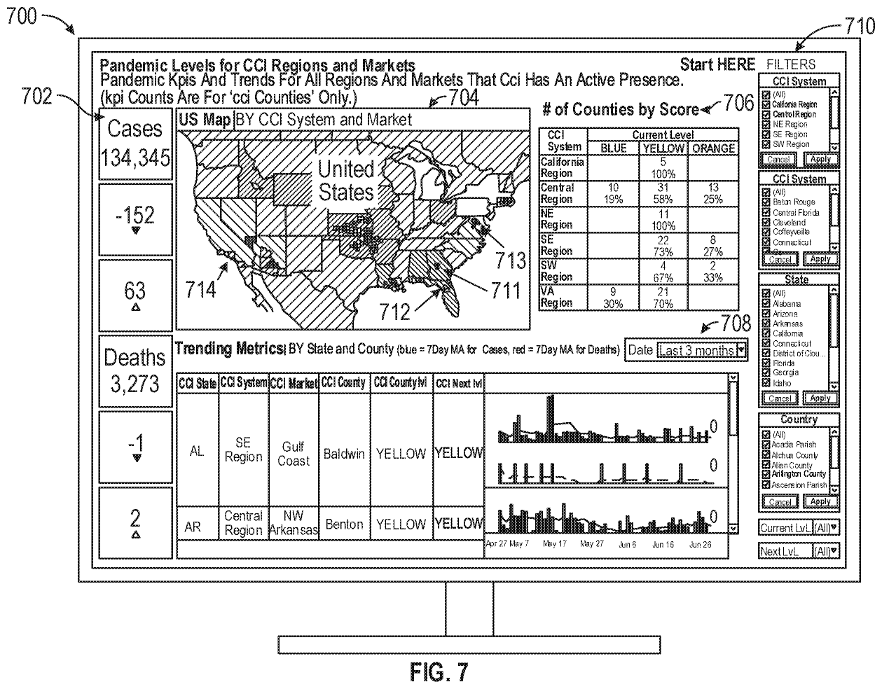

FIG. 7 depicts another workplace disruption dashboard 700. The dashboard 700 may provide information associated with particular regions of interest (for example, region of interest 711, region of interest 712, region of interest 713, region of interest 714, and/or any other number of regions of interest) as depicted in the map 704 rather than the general geographical regions that may be depicted in dashboards 200-500 as described above. For example, a place of business may include several regions of operation throughout the country, and the map 704 may only present information associated with those very particular regions of operation. In some cases, information regarding the location of the regions of operation may be manually provided, or may automatically be obtained from a data source, such as a server. The dashboard 700 may also provide one or more filters 710. A user may be able to select some or all of the one or more filters 710 in order to adjust the regions for which information is displayed on the map 704. For example, if a particular place of business has regions of operation in Georgia and Florida, a user may select a filter for Georgia and may deselect a filter for Florida. In this particular example, the map 704 may then only present information associated with operating regions within Georgia. The one or more filters 710 may also allow a user to filter by any other criteria other than geographical region, such as by various metrics or any other filterable criteria. In some cases, similar to other dashboards described herein, the dashboard 700 may present various metrics relating to the particular regions of interest indicated through the one or more filtered 710 that are selected. For example, the dashboard 700 may present a first set of metrics 706 associated with the regions selected using the one or more filters 710. For example, as depicted in the dashboard 700, the first set of metrics 706 may include a current level for the different regions selected using the one or more filters 710. In some cases, the dashboard 700 may also present a second set of metrics 708. The second set of metrics 708 may, for example, present additional information that is more specific to different regions selected through the one or more filters 710. For example, the second set of metrics 708 may indicate a state, a region, a county, a current level, a predicted next level, and/or a plot illustrating a trend over a given period of time. The dashboard 700 may also present any other relevant metrics in any other format as well.

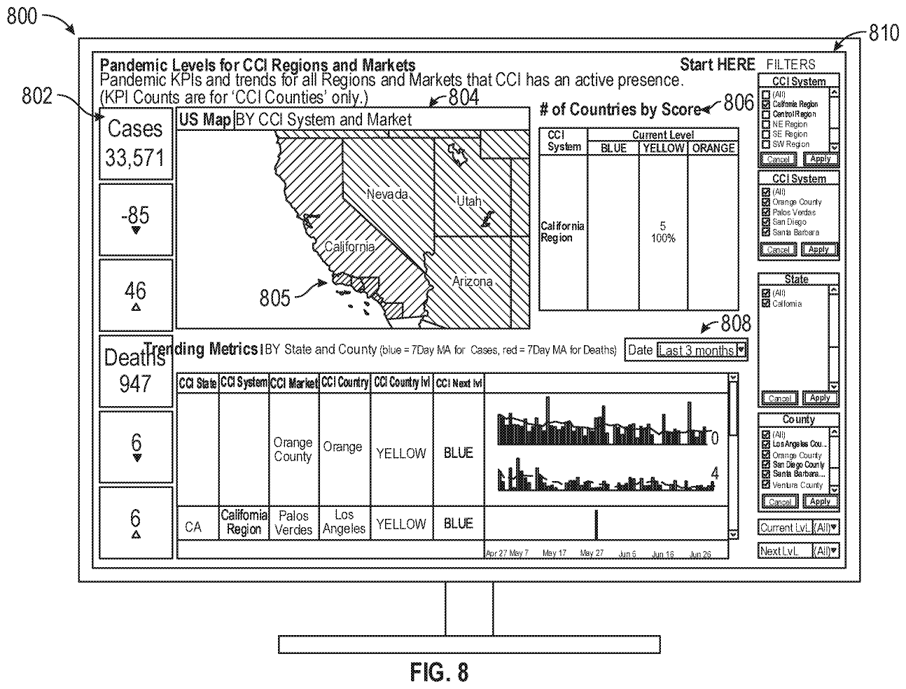

FIG. 8 depicts another workplace disruption dashboard 800. The dashboard 800 may be similar to dashboard 700, but may illustrate a map 804 that may be presented when only some of the one or more filters 810 are selected by a user. The dashboard 800 may provide a specific example of a user selecting a California region from the one or more filters 810 (with all of the other regions being de-selected). Accordingly, the map 804 presents a zoomed-in version of the map 704 depicted in FIG. 7. Particularly, the map is zoomed-in such that the focus of the map 804 is the particular regions of interest 705 associated with the California region. As depicted in the figure, this region of interest may not necessarily include the entire state of California, but may rather be associated with a sub-region of the state. For example, a place of business may indicate one or more operating regions within the state of California, and these operating regions may be the focus of the map 804 when the user selects a filter associated with the California region. Additionally, any of the metrics presented in the dashboard 800 may automatically adjust as the user selects and/or de-deselects any of the filters 810. For example, if the user selects only the California region as illustrated in the figure, the metrics may automatically adjust to only reflect that particular region. If the user were also to select another region, the metrics may be adjusted to include information associated with both regions as a whole, or alternatively may include metrics associated with each individual region as well. The dashboard 800 may also be configured to present metrics for the individual regions simultaneously for comparison purposes as well.

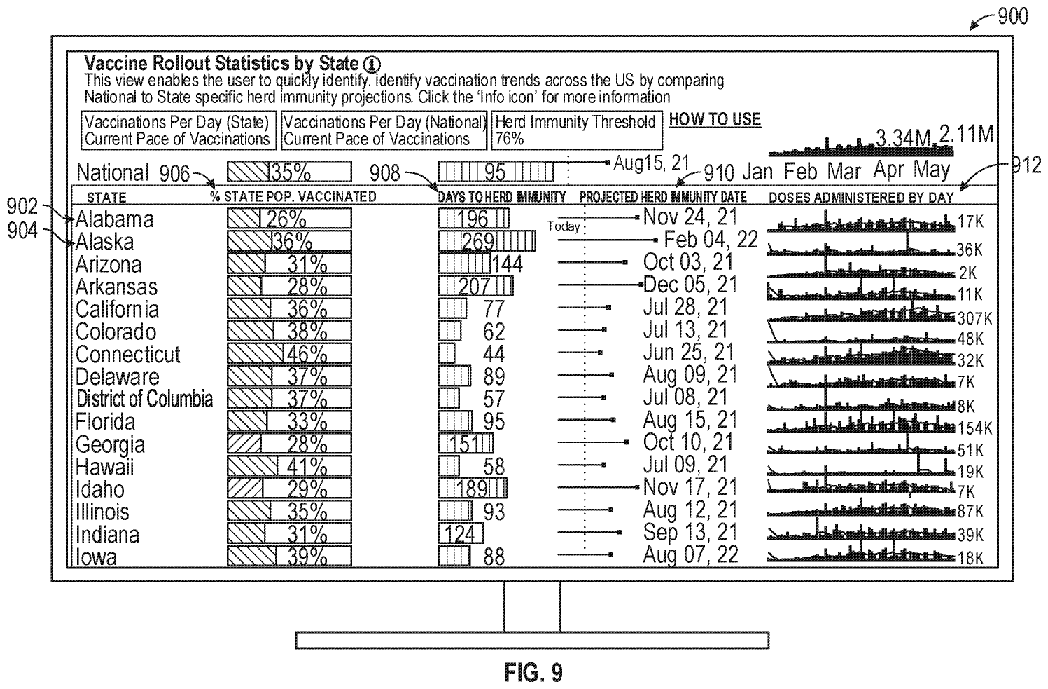

FIG. 9 depicts another workplace disruption dashboard 900. The workplace disruption dashboard 900 may illustrate information relating to vaccination rollout statistics (this particular workplace disruption dashboard 900, as well as the dashboards 1000-1300, may be specific to pandemic disruption events). The dashboard 900 may present information associated with different geographical regions (for example, a first state 902, a second state 904, and/or any other number of geographical regions). The information may include a first metric 906, a second metric 908, a third metric 910, and/or any other number of metrics. The information may also include a visual depiction of some or all of the information, such as a plot 912. In the particular example depicted in FIG. 9, the first metric 906 may include a percentage of a population of the region that has been vaccinated. The second metric 908 may include a number of days until a “herd immunity” is achieved. The third metric 910 may include a projected herd immunity date.

In some embodiments, the projected herd immunity date (as well as any other forecasted metrics described herein or otherwise) may be determined using one or more predictive algorithms. In some cases, the predictive algorithms may employ artificial intelligence, machine learning, and/or the like. In some cases, the artificial intelligence, machine learning, and/or the like may be pre-trained before being implemented to perform real-time predictions. The pre-implementation training may be performed by providing input data to the predictive algorithm, while also indicating what the corresponding output(s) should be for the given input data. Additionally, the artificial intelligence, machine learning, and/or the like may also be continuously trained even after being implemented as well. That is, the artificial intelligence, machine learning, and/or the like may be pre-trained before being implemented to perform predictions, but may continue to be trained while analyzing real-time data associated with disruptive events. In this manner, the artificial intelligence may become more effective at forecasting metrics associated with the disruptive events. Additionally, in some cases, the predictive algorithms may rely on Bayesian structural equations or any other types of statistical analyses.

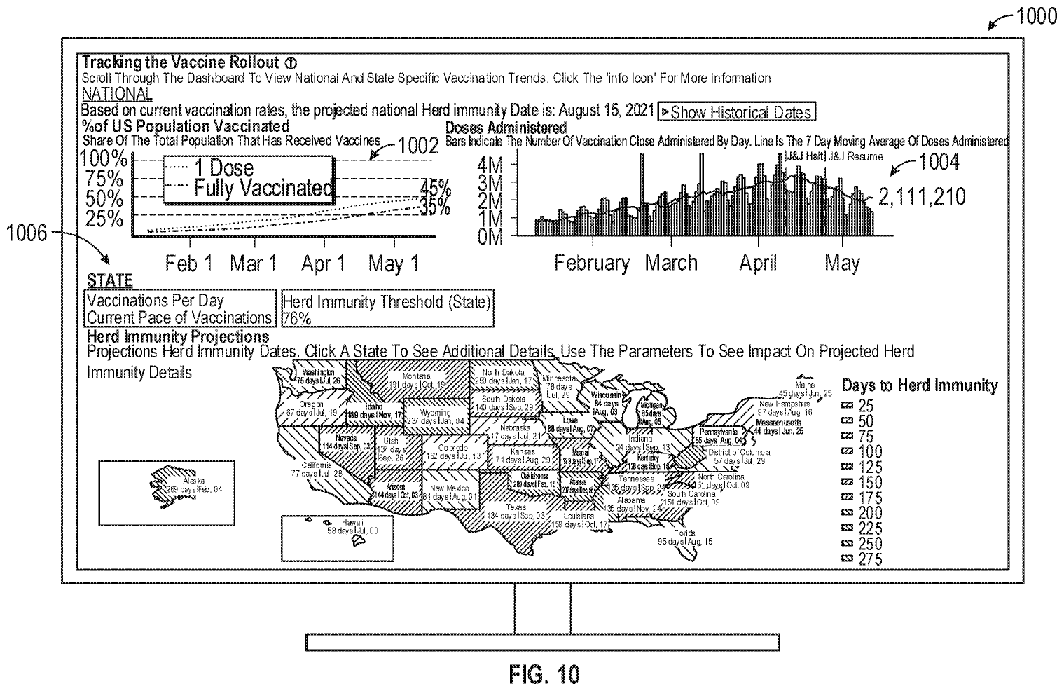

FIG. 10 depicts another workplace disruption dashboard 1000. The dashboard 1000 may present additional information pertaining to a vaccine rollout. For example, the dashboard 1000 may present one or more plots visualizing different types of information relating to the vaccine rollout. A first example plot 1002 may visualize trends relating to a percentage of a population that has been vaccinated. A second example plot 1004 may visualize trends associated with a number of vaccine doses that are administered. As another example, the dashboard 1000 may present a map 1006 including herd immunity projections for different regions based on vaccine data. For example, the map 1006 may be a map of the United States and may include herd immunity projection data for each of the individual states. The herd immunity projection data may include a number of days until herd immunity, a predicted herd immunity date, and/or any other data. The individual states may also be provided different colors or shading to indicate which of the states are closer to a herd immunity date than other states.

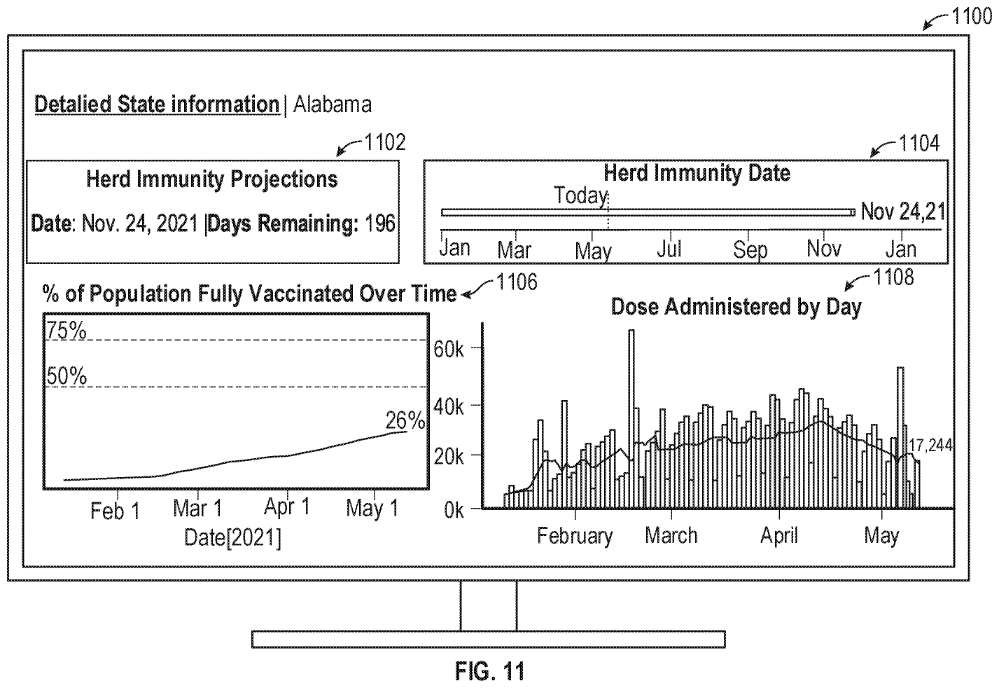

FIG. 11 depicts another workplace disruption dashboard 1100. The dashboard 1100 may be similar to dashboard 1000, but may present information for sub-regions (for example, sub-regions of the map 1006 presented in FIG. 10). That is, the dashboard 1000 may allow a user to select a particular sub-region of a map, and information specific to that sub-region may be presented on the dashboard 1100. Continuing the example of the dashboard 1000 in FIG. 10, a user may select a particular state on the map, and vaccine information specific to that state may then be presented in the dashboard 1100. This information may include at least herd immunity projection information 1102, a timeline for herd immunity 1104, a plot 1106 depicting a trend relating to the percentage of the population of the sub-region that has been vaccinated, and/or a plot 1108 depicting a trend relating to a number of vaccine doses administered over a given period of time.

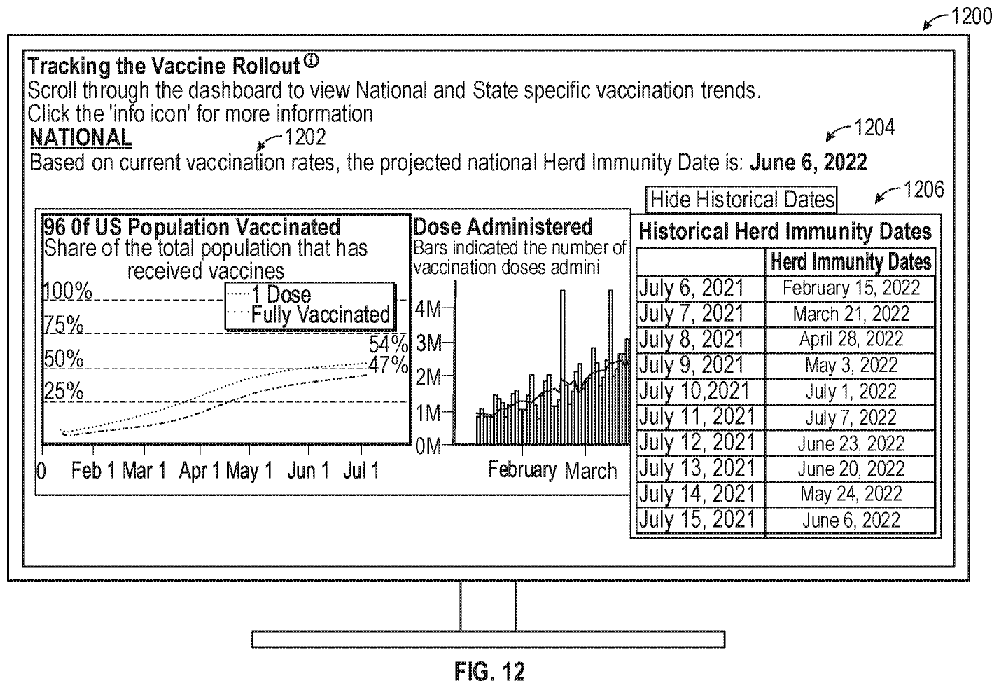

FIG. 12 depicts another workplace disruption dashboard 1200. The dashboard 1200 may depict some similar elements as dashboard 1000. That is, a first example plot 1202 may be the same as first example plot 1002, and a second example plot 1204 may be the same as second example plot 1004. The dashboard 1200 may illustrate that any of the example plots depicted in the dashboard 100 may be interacted with such that a user may be able to receive more specific information by interacting with any of the plots. For example, as depicted in FIG. 12, a user may select a portion of the second example plot 1204, which may result in a separate interface 1206 being presented on top of the dashboard 1200. The separate interface 1206 in this particular example may present information relating to a predicted herd immunity date for every day over a given time range. That is, the predicted herd immunity date may change depending on data, such as a number of doses of vaccines that are administered on any given day.

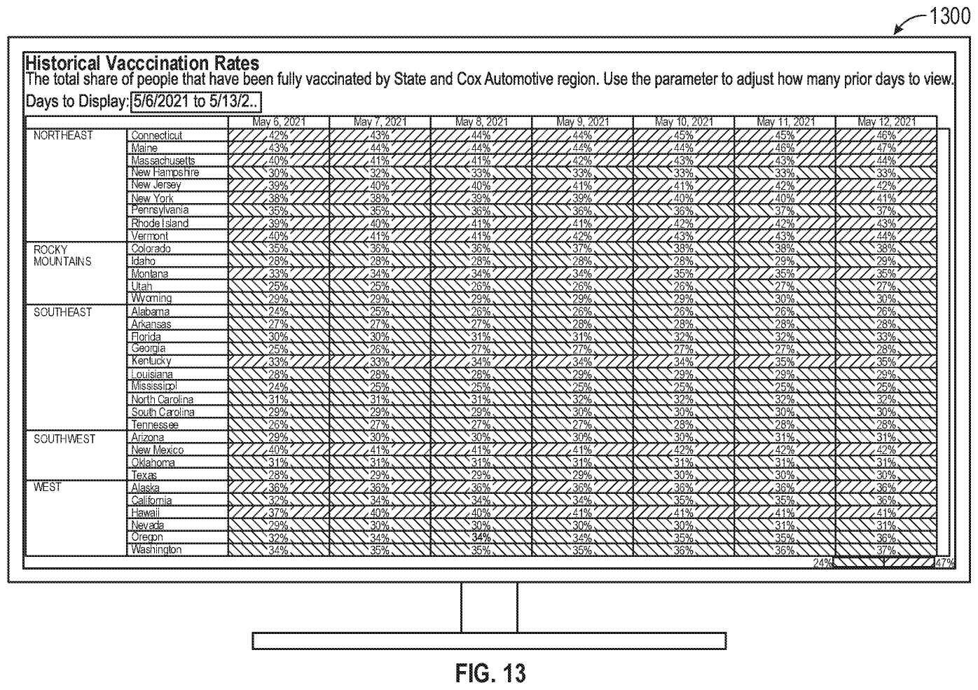

FIG. 13 depicts another workplace disruption dashboard 1300. The dashboard 1300 may present additional vaccination metrics. Particularly, the dashboard 1300 may present historical vaccination rates for different regions.

FIG. 14 depicts an illustrative table 1400 of work disruption levels, in accordance with one or more example embodiments of the present disclosure. A work disruption level is marked by a red color/score indicative of a county having an extremely severe disruption and which protocols may be implemented as a result of the disruption, an orange color/score indicative of a county having a moderately severe disruption, a yellow color/score indicative of a county having a mildly severe disruption, and a blue color/score indicative of a county having a non-severe disruption. For any work disruption level/score, actions defined by workplace protocols are recommended, e.g., how many and/or what types of employees who may work at the workplace or may work from home, which buildings may be closed, whether or not international or domestic travel may be permitted and may require particular levels of supervisory permission, and/or how many people may be allowed to gather. When a geographic area has or is projected to have a workplace disruption score, the corresponding protocols may be sent automatically, using a computer system, to one or more other computer systems, such as travel booking systems, office management systems, and the like, based on the workplace restrictions specified by the protocols that correspond to the disruption score/level. For example, a disruption score may fall into a work disruption level based on the thresholds that define the levels (e.g., score ranges).

FIG. 15 depicts a table 1500 of an example of work disruption protocols, in accordance with one or more example embodiments of the present disclosure. As shown in FIG. 3, workplace disruption levels are applied to multiple types of workplaces, for example, as defined by the types of employees or other stakeholders allowed on workplace premises. For any workplace disruption level, protocols govern whether or not that type of employees may WFH, stay at home, and/or travel locally or non-locally (e.g., outside of a metropolitan area). For example, one type of workplace category may not have any on-site client-customer interaction, and the number of employees recommended to work from home may be different than a workplace that has on-site customer-client interaction. Work from home recommendations may depend on the disruption score and the ability of employees to work from home (e.g., based on the type of work at the workplace, such as desk work or manual labor).

FIG. 16 depict example tables 1600 of work disruption levels and corresponding procedural guidelines, such as which facilities may be opened/closed, which portions of facilities may be available, requirements for protective equipment to be worn by employees and/or non-employee visitors, and the like, in accordance with one or more example embodiments of the present disclosure. As shown in FIG. 16, workplace disruption levels may correspond to protocols governing facilities, face covering and other personal protective equipment, etc. to indicate which protocols may be applied, which facilities may be closed, and which personal protective equipment may be required to be worn at a workplace, etc.

FIG. 17 depicts an example table 1700 of work disruption levels and corresponding guidelines for workplace visitors and contractors, in accordance with one or more example embodiments of the present disclosure. As shown in FIG. 17, workplace disruption levels indicate that which types of people (e.g., customers, vendors, contractors, and/or visitors) may be allowed to access a particular facility, and/or particular areas of a facility (e.g., some physical areas may be more restricted than others).

FIG. 18 depicts example tables 1800 of travel restrictions, in accordance with one or more example embodiments of the present disclosure. As shown in FIG. 6, workplace disruption levels indicate that which types of travel (e.g., emergency travel and/or critical business travel) approved by which level of managers (e.g., senior vice president, vice president, and/or director) may be permitted at a particular departure geographical location and/or a destination location.

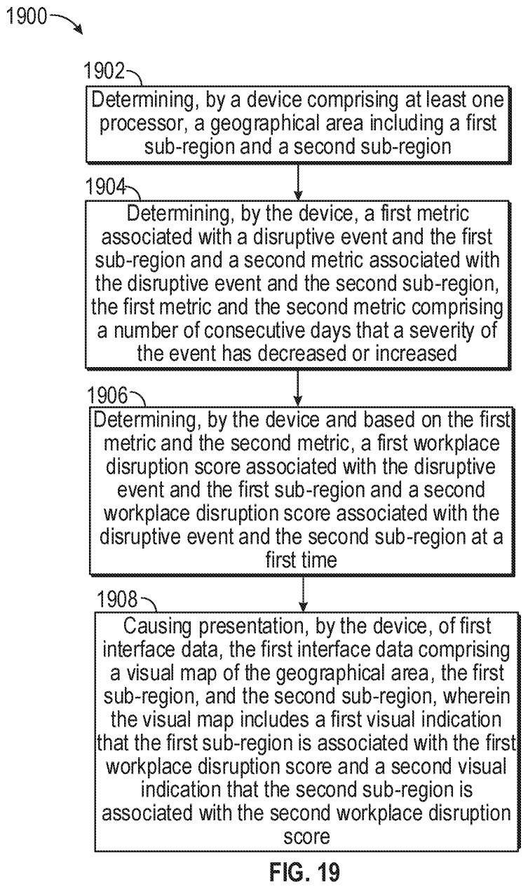

FIG. 19 depicts a flow diagram of an illustrative process 1900 for generating a work disruption dashboard, in accordance with one or more embodiments of the disclosure.

At block 1902, a device may determine a geographical area including a first sub-region and a second sub-region.

At block 1904, the device may determine a first metric associated with a disruptive event and the first sub-region and a second metric associated with the disruptive event and the second sub-region, the first metric and the second metric comprising a number of consecutive days that a severity of the event has decreased or increased. For instance, a disruptive event may be an event disrupting a workplace. Examples of an event may include natural disasters (e.g., hurricanes, earthquakes, and pandemics, or the like), manmade disasters (e.g., drastic changes in economic conditions and geopolitical tensions leading to widespread labor unrest and war, or the like), an event caused by bad weather, an event caused by massive rally, or any other event relevant to a workplace disruption. Examples of metrics may include a number of total cases (e.g., a number of infected/ill people) associated with a disruptive event in a particular area, a number of newly added cases (e.g., a number of infected/ill people) associated with the disruptive event in the particular area, a number of total death cases associated with the disruptive event in the particular area, severity ranges associated with the particular area, a number of declining cases associated, previous and future weather patterns, actual and projected event crowd sizes, and/or any metric indicative of a workplace disruption in a particular area, as shown in FIG. 1A-FIG. 1D. The device may determine the metrics at one or more levels (e.g., country-level, state-level, region-level, county-level, city-level, etc.).

At block 1906, the device may determine, based on the first metric and the second metric, a first workplace disruption score associated with the disruptive event and the first sub-region and a second workplace disruption score associated with the disruptive event and the second sub-region at a first time. For instance, a workplace disruption score may be indicative of a current level of disruption or threat of disruption caused by the disruptive event, such as the severity of a virus or illness (e.g., the number of people who contract the virus or illness, the morbidity rate of a virus or illness, etc.), the severity of a weather disruption, the number of road or other transportation closures caused by the disruptive event, and the like. Based on the workplace disruption score, a workplace protocol may be presented when a user of the device selects (e.g., explicitly or implicitly through a preference or user history) an area or sub-area to view. In some embodiments, the device may determine a disruption score indicative of a current level of disruption or threat of disruption caused by the disruptive event.

At block 1908, the device may cause presentation of first interface data, the first interface data comprising a visual map of the geographical area, the first sub-region, and the second sub-region, wherein the visual map includes a first visual indication that the first sub-region is associated with the first workplace disruption score and a second visual indication that the second sub-region is associated with the second workplace disruption score. For instance, a computer system may generate a workplace disruption dashboard (e.g., 100-1300 of FIGS. 1-13) to present a map that indicates the disruption scores at a geographical scale (e.g., at a county scale, at a zip code scale, at a state scale, a city scale, at an area code scale, and/or at any other scale associated with a boundary area). The computer system may present the geographic areas with their respective disruption scores (e.g., using a color-coded map whose colors/shades indicate the disruption scores). When a user of the computer system selects a geographic area (e.g., by hovering over the area, clicking the area, touching the area, etc.), the computer system may present one or more additional graphical user interfaces concurrently or in place of the map interface to present a customized display of data relevant to the disruption score, including a projected change in the score (e.g., the score may increase or decrease in a number of days based on an increasing or decreasing trend of severity indicated by the metrics for an area or sub-area). Examples are described in FIGS. 1-13.

It is understood that the above descriptions are for purposes of illustration and are not meant to be limiting.

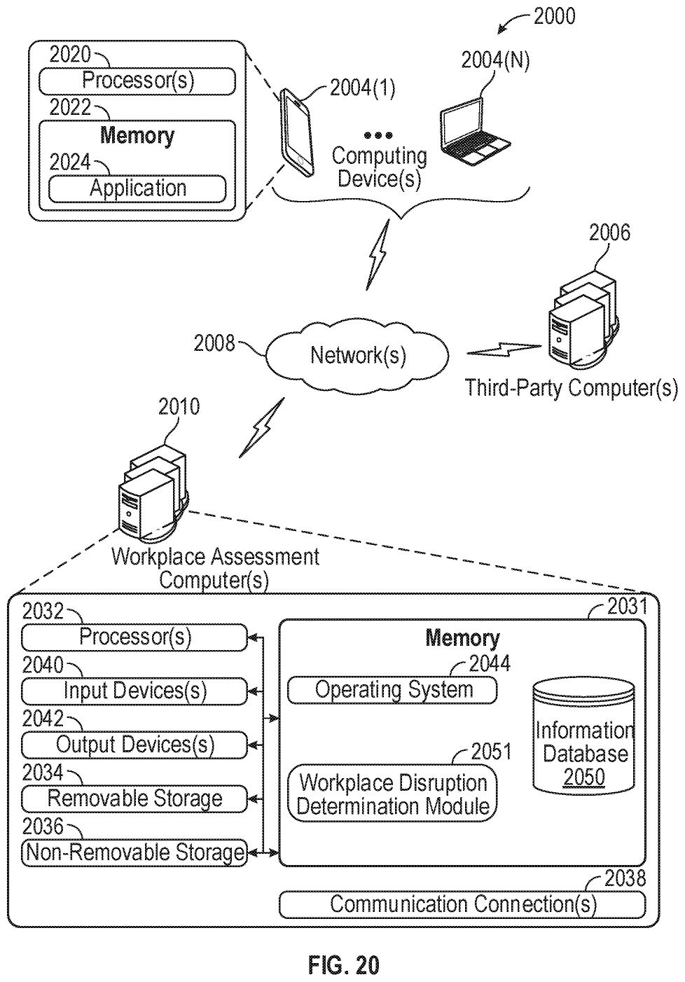

FIG. 20 depicts a diagram illustrating an example network environment for generating a workplace disruption dashboard, in accordance with one or more example embodiments of the present disclosure. As shown in FIG. 20, a system 2000 may include one or more workplace assessment computers 2010, one or more computing devices 2004(1), . . . , 2004(N) and one or more third-party computers 2006. In the system 2000, users may utilize the computing devices 2004 to access an application interface 2030 that may be provided by, created by, or otherwise associated with the workplace assessment computers 2010 via one or more networks 2008. The one or more computing devices 2004(1), . . . , 2004(N) may call one or more active programming interfaces of the one or more workplace assessment computers 2010 using the application interface 2030 to provide a workplace disruption dashboard. In some instances, the computing devices 2004 may be configured to present or otherwise display the application interface 2030 to the users. While the illustrated example represents the users accessing the application interface 2030 over the networks 2008, the described techniques may equally apply in instances where the users interact with the workplace disruption dashboard via a personal computer, over the phone, via a kiosk, or in any other manner. It is also noted that the described techniques may apply in other client/server arrangements (e.g., set-top boxes, etc.), as well as in non-client/server arrangements (e.g., locally stored software applications, etc.).

In some aspects, the application interface 2030 associated with the computing devices 2004 may allow the users to access, receive from, transmit to, or otherwise interact with workplace assessment computers 2010. In some examples, the application interface 2030 may also allow the users to transmit to the workplace assessment computers 2010 over the networks 2008 information associated with one or more workplaces.

The workplace assessment computers 2010 may be any type of computing devices, such as, but not limited to, mobile, desktop, and/or cloud computing devices, such as servers. In some examples, the workplace assessment computers 2010 may be in communication with the computing devices 2004 and the third party computers 2006 via the networks 2008, or via other network connections. The workplace assessment computers 2010 may include one or more servers, perhaps arranged in a cluster, as a server farm, or as individual servers not associated with one another. These servers may be configured to host a website viewable via the application interface 2030 associated with the computing devices 2004 or any other Web browser accessible by a user. In addition, the workplace assessment computers 2010 may communicate with one or more applications or other programs running the computing devices 2004.

The computing devices 2004 may be any type of computing devices including, but not limited to, desktop personal computers (PCs), laptop PCs, mobile phones, smartphones, personal digital assistants (PDAs), tablet PCs, game consoles, set-top boxes, wearable computers, e-readers, web-enabled TVs, cloud-enabled devices and work stations, and the like. In certain aspects, the computing devices 2004 may include touch screen capabilities, motion tracking capabilities, cameras, microphones, vision tracking, etc. In some instances, each computing device 204 may be equipped with one or more processors 2020 and memory 2022 to store applications and data, such as an auction application 2024 that may display the client application interface 2030 and/or enable access to a website stored on the workplace assessment computers 2010, or elsewhere, such as a cloud computing network.

The third-party computers 2006 may also be any type of computing devices such as, but not limited to, mobile, desktop, and/or cloud computing devices, such as servers. In some examples, the third-party computers 2006 may be in communication with the workplace assessment computers 2010 and/or the computing devices 2004 via the networks 2008, or via other network connections. The third-party computers 2006 may include one or more servers, perhaps arranged in a cluster, as a server farm, or as individual servers not associated with one another. These servers may be configured to provide information associated with a disruptive event.

In one illustrative configuration, the workplace assessment computer 2010 may include at least a memory 2031 and one or more processing units (or processors) 2032. The processors 2032 may be implemented as appropriate in hardware, software, firmware, or combinations thereof. Software or firmware implementations of the processors 2032 may include computer-executable or machine-executable instructions written in any suitable programming language to perform the various functions described.

The memory 2031 may store program instructions that are loadable and executable on the processors 2032, as well as data generated during the execution of these programs. Depending on the configuration and type of workplace assessment computer 2010, the memory 2031 may be volatile (such as random access memory (RAM)) and/or non-volatile (such as read-only memory (ROM), flash memory, etc.). The workplace assessment computer 2010 or server may also include additional removable storage 2034 and/or non-removable storage 2036 including, but not limited to, magnetic storage, optical disks, and/or tape storage. The disk drives and their associated computer-readable media may provide non-volatile storage of computer-readable instructions, data structures, program modules, and other data for the computing devices. In some implementations, the memory 2031 may include multiple different types of memory, such as static random access memory (SRAM), dynamic random access memory (DRAM), or ROM.

The memory 2031, the removable storage 2034, and the non-removable storage 2036 are all examples of computer-readable storage media. For example, computer-readable storage media may include volatile and non-volatile, removable and non-removable media implemented in any method or technology for the storage of information such as computer-readable instructions, data structures, program modules, or other data. The memory 2031, the removable storage 2034, and the non-removable storage 2036 are all examples of computer storage media. Additional types of computer storage media that may be present include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the desired information and which can be accessed by the workplace assessment computer 2010 or other computing devices. Combinations of the any of the above should also be included within the scope of computer-readable media.

Alternatively, computer-readable communication media may include computer-readable instructions, program modules, or other data transmitted within a data signal, such as a carrier wave, or other transmission. However, as used herein, computer-readable storage media does not include computer-readable communication media.

The workplace assessment computer 2010 may also contain communication connection(s) 2038 that allow the workplace assessment computer 2010 to communicate with a stored database, another computing device or server, user terminals, and/or other devices on a network. The workplace assessment computer 2010 may also include input device(s) 2040 such as a keyboard, a mouse, a pen, a voice input device, a touch input device, etc., and output device(s) 2042, such as a display, speakers, printers, etc.

Turning to the contents of the memory 2031 in more detail, the memory 2031 may include an operating system 2044 and one or more application programs or services for implementing the features disclosed herein, including a workplace disruption determination module 2051. In some instances, the workplace disruption determination module 2051 may receive, transmit, and/or store information in the database 2050.

The workplace disruption determination module 2051 may generate a workplace disruption dashboard to present a map that indicates workplace disruption scores at a geographical scale (e.g., at a county scale, at a zip code scale, at a state scale, a city scale, at an area code scale, and/or at any other scale associated with a boundary area). The workplace disruption determination module 2051 may identify geographic areas, determine respective workplace disruption scores for the geographic areas, and may present the geographic areas with their respective disruption scores (e.g., using a color-coded map whose colors indicate the disruption scores). The workplace disruption determination module 2051 may generate one or more graphical user interfaces to present the geographic areas and their respective disruption scores. When a user selects a geographic area (e.g., by hovering over the area, clicking the area, touching the area, etc.), the workplace disruption determination module 2051 may generate one or more additional graphical user interfaces concurrently or in place of the map interface to present a customized display of data relevant to the disruption score, including a projected change in the score (e.g., the score will increase or decrease in a number of days). Examples are described in FIGS. 1-13.

The computing devices 2004, the one or more third-party computers 2006, and the one or more workplace assessment computers 2010 may be configured to communicate via the one or more networks 2008, wirelessly or wired. The one or more networks 2008 may include, but not limited to, any one of a combination of different types of suitable communications networks such as, for example, broadcasting networks, cable networks, public networks (e.g., the Internet), private networks, wireless networks, cellular networks, or any other suitable private and/or public networks. Further, the one or more networks 2008 may have any suitable communication range associated therewith and may include, for example, global networks (e.g., the Internet), metropolitan area networks (MANs), wide area networks (WANs), local area networks (LANs), or personal area networks (PANs). In addition, the one or more networks 2008 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, white space communication mediums, ultra-high frequency communication mediums, satellite communication mediums, or any combination thereof.

Various instructions, methods, and techniques described herein may be considered in the general context of computer-executable instructions, such as program modules, executed by one or more computers or other devices. Generally, program modules may include routines, programs, objects, components, data structures, etc., for performing particular tasks or implementing particular abstract data types. These program modules and the like may be executed as native code or may be downloaded and executed, such as in a virtual machine or other just-in-time compilation execution environment. Typically, the functionality of the program modules may be combined or distributed as desired in various embodiments. An implementation of these modules and techniques may be stored on some form of computer-readable storage media.

The example architectures and computing devices shown in FIG. 20 are provided by way of example only. Numerous other operating environments, system architectures, and device configurations are possible. Accordingly, embodiments of the present disclosure should not be construed as being limited to any particular operating environment, system architecture, or device configuration.

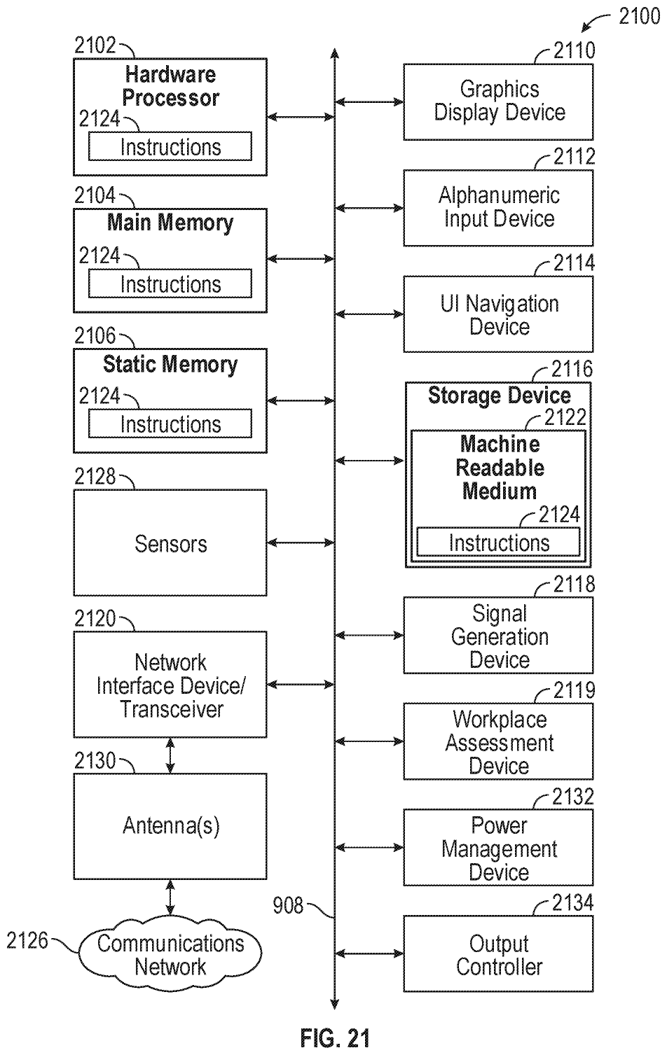

FIG. 21 depicts a block diagram of an example machine 2100 upon which any of one or more techniques (e.g., methods) may be performed, in accordance with one or more example embodiments of the present disclosure. In other embodiments, the machine 2100 may operate as a standalone device or may be connected (e.g., networked) to other machines. In a networked deployment, the machine 2100 may operate in the capacity of a server machine, a client machine, or both in server-client network environments. In an example, the machine 2100 may act as a peer machine in peer-to-peer (P2P) (or other distributed) network environments. The machine 2100 may be a personal computer (PC), a tablet PC, a set-top box (STB), a personal digital assistant (PDA), a mobile telephone, a wearable computer device, a web appliance, a network router, a switch or bridge, or any machine capable of executing instructions (sequential or otherwise) that specify actions to be taken by that machine, such as a base station. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), or other computer cluster configurations.

Examples, as described herein, may include or may operate on logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In another example, the hardware may include configurable execution units (e.g., transistors, circuits, etc.) and a computer readable medium containing instructions where the instructions configure the execution units to carry out a specific operation when in operation. The configuring may occur under the direction of the executions units or a loading mechanism. Accordingly, the execution units are communicatively coupled to the computer-readable medium when the device is operating. In this example, the execution units may be a member of more than one module. For example, under operation, the execution units may be configured by a first set of instructions to implement a first module at one point in time and reconfigured by a second set of instructions to implement a second module at a second point in time.

The machine (e.g., computer system) 2100 may include a hardware processor 2102 (e.g., a central processing unit (CPU), a graphics processing unit (GPU), a hardware processor core, or any combination thereof), a main memory 2104 and a static memory 2106, some or all of which may communicate with each other via an interlink (e.g., bus) 2108. The machine 2100 may further include a power management device 2132, a graphics display device 2110, an alphanumeric input device 2112 (e.g., a keyboard), and a user interface (UI) navigation device 2114 (e.g., a mouse). In an example, the graphics display device 2110, alphanumeric input device 2112, and UI navigation device 2114 may be a touch screen display. The machine 2100 may additionally include a storage device (i.e., drive unit) 2116, a signal generation device 2118 (e.g., a speaker), a work assessment device 2119, a network interface device/transceiver 2120 coupled to antenna(s) 2130, and one or more sensors 2128, such as a global positioning system (GPS) sensor, a compass, an accelerometer, or other sensor. The machine 2100 may include an output controller 2134, such as a serial (e.g., universal serial bus (USB), parallel, or other wired or wireless (e.g., infrared (IR), near field communication (NFC), etc.) connection to communicate with or control one or more peripheral devices (e.g., a printer, a card reader, etc.)).

The storage device 2116 may include a machine readable medium 2122 on which is stored one or more sets of data structures or instructions 2124 (e.g., software) embodying or utilized by any one or more of the techniques or functions described herein. The instructions 2124 may also reside, completely or at least partially, within the main memory 2104, within the static memory 2106, or within the hardware processor 2102 during execution thereof by the machine 2100. In an example, one or any combination of the hardware processor 2102, the main memory 2104, the static memory 2106, or the storage device 2116 may constitute machine-readable media.

The work assessment device 2119 may carry out or perform any of the operations and processes (e.g., process 700 of FIG. 7) described and shown above, and may facilitate the analysis and display of workplace disruption metrics, the display of workplace disruption scores and related data, the projected changes to workplace disruption scores, protocols governing workplaces, and the like.

It is understood that the above are only a subset of what the work assessment device 2119 may be configured to perform and that other functions included throughout this disclosure may also be performed by the work assessment device 2119.

While the machine-readable medium 2122 is illustrated as a single medium, the term “machine-readable medium” may include a single medium or multiple media (e.g., a centralized or distributed database, and/or associated caches and servers) configured to store the one or more instructions 2124.

Various embodiments may be implemented fully or partially in software and/or firmware. This software and/or firmware may take the form of instructions contained in or on a non-transitory computer-readable storage medium. Those instructions may then be read and executed by one or more processors to enable performance of the operations described herein. The instructions may be in any suitable form, such as but not limited to source code, compiled code, interpreted code, executable code, static code, dynamic code, and the like. Such a computer-readable medium may include any tangible non-transitory medium for storing information in a form readable by one or more computers, such as but not limited to read only memory (ROM); random access memory (RAM); magnetic disk storage media; optical storage media; a flash memory, etc.

The term “machine-readable medium” may include any medium that is capable of storing, encoding, or carrying instructions for execution by the machine 2100 and that cause the machine 2100 to perform any one or more of the techniques of the present disclosure, or that is capable of storing, encoding, or carrying data structures used by or associated with such instructions. Non-limiting machine-readable medium examples may include solid-state memories and optical and magnetic media. In an example, a massed machine-readable medium includes a machine-readable medium with a plurality of particles having resting mass. Specific examples of massed machine-readable media may include non-volatile memory, such as semiconductor memory devices (e.g., electrically programmable read-only memory (EPROM), or electrically erasable programmable read-only memory (EEPROM)) and flash memory devices; magnetic disks, such as internal hard disks and removable disks; magneto-optical disks; and CD-ROM and DVD-ROM disks.

The instructions 2124 may further be transmitted or received over a communications network 2126 using a transmission medium via the network interface device/transceiver 2120 utilizing any one of a number of transfer protocols (e.g., frame relay, internet protocol (IP), transmission control protocol (TCP), user datagram protocol (UDP), hypertext transfer protocol (HTTP), etc.). Example communications networks may include a local area network (LAN), a wide area network (WAN), a packet data network (e.g., the Internet), mobile telephone networks (e.g., cellular networks), plain old telephone (POTS) networks, wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, and peer-to-peer (P2P) networks, among others. In an example, the network interface device/transceiver 2120 may include one or more physical jacks (e.g., Ethernet, coaxial, or phone jacks) or one or more antennas to connect to the communications network 2126. In an example, the network interface device/transceiver 2120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 2100 and includes digital or analog communications signals or other intangible media to facilitate communication of such software. The operations and processes described and shown above may be carried out or performed in any suitable order as desired in various implementations. Additionally, in certain implementations, at least a portion of the operations may be carried out in parallel. Furthermore, in certain implementations, less than or more than the operations described may be performed.

Some embodiments may be used in conjunction with various devices and systems, for example, a personal computer (PC), a desktop computer, a mobile computer, a laptop computer, a notebook computer, a tablet computer, a server computer, a handheld computer, a handheld device, a personal digital assistant (PDA) device, a handheld PDA device, an on-board device, an off-board device, a hybrid device, a vehicular device, a non-vehicular device, a mobile or portable device, a consumer device, a non-mobile or non-portable device, a wireless communication station, a wireless communication device, a wireless access point (AP), a wired or wireless router, a wired or wireless modem, a video device, an audio device, an audio-video (A/V) device, a wired or wireless network, a wireless area network, a wireless video area network (WVAN), a local area network (LAN), a wireless LAN (WLAN), a personal area network (PAN), a wireless PAN (WPAN), and the like.

Some embodiments may be used in conjunction with one way and/or two-way radio communication systems, cellular radio-telephone communication systems, a mobile phone, a cellular telephone, a wireless telephone, a personal communication system (PCS) device, a PDA device which incorporates a wireless communication device, a mobile or portable global positioning system (GPS) device, a device which incorporates a GPS receiver or transceiver or chip, a device which incorporates an RFID element or chip, a multiple input multiple output (MIMO) transceiver or device, a single input multiple output (SIMO) transceiver or device, a multiple input single output (MISO) transceiver or device, a device having one or more internal antennas and/or external antennas, digital video broadcast (DVB) devices or systems, multi-standard radio devices or systems, a wired or wireless handheld device, e.g., a smartphone, a wireless application protocol (WAP) device, or the like.

Some embodiments may be used in conjunction with one or more types of wireless communication signals and/or systems following one or more wireless communication protocols, for example, radio frequency (RF), infrared (IR), frequency-division multiplexing (FDM), orthogonal FDM (OFDM), time-division multiplexing (TDM), time-division multiple access (TDMA), extended TDMA (E-TDMA), general packet radio service (GPRS), extended GPRS, code-division multiple access (CDMA), wideband CDMA (WCDMA), CDMA 2000, single-carrier CDMA, multi-carrier CDMA, multi-carrier modulation (MDM), discrete multi-tone (DMT), Bluetooth®, global positioning system (GPS), Wi-Fi, Wi-Max, ZigBee, ultra-wideband (UWB), global system for mobile communications (GSM), 2G, 2.5G, 3G, 3.5G, 4G, fifth generation (5G) mobile networks, 3GPP, long term evolution (LTE), LTE advanced, enhanced data rates for GSM Evolution (EDGE), or the like. Other embodiments may be used in various other devices, systems, and/or networks.

What is claimed is:

1. A method comprising:
determining, by a device comprising at least one processor, a geographical area including a first sub-region and a second sub-region;
determining, by the device, a first metric associated with a disruptive event and the first sub-region and a second metric associated with the disruptive event and the second sub-region, the first metric and the second metric comprising a number of consecutive days that a severity of the event has decreased or increased;
determining, by the device and based on the first metric and the second metric, a first workplace disruption score associated with the disruptive event and the first sub-region and a second workplace disruption score associated with the disruptive event and the second sub-region at a first time; and
causing presentation, by the device, of first interface data, the first interface data comprising a visual map of the geographical area, the first sub-region, and the second sub-region, wherein the visual map includes a first visual indication that the first sub-region is associated with the first workplace disruption score and a second visual indication that the second sub-region is associated with the second workplace disruption score.

2. The method of claim 1, further comprising:
determining that the first workplace disruption score is greater than a first threshold value; and
automatically sending a notification to one or more users associated with the first sub-region based on the determination that the first workplace disruption score is greater than a first threshold value.

3. The method of claim 1, wherein the disruptive event is a pandemic event, and wherein the method further comprises:
predicting, using a Bayesian structural equation and based on the first metric, a herd immunity date for the first sub-region.

4. The method of claim 1, further comprising:
determining, by the device, a third workplace disruption score associated with the disruptive event and the first sub-region at a second time after the first time; and
causing presentation, by the device, of second interface data, the second interface data comprising the geographical area and a third visual indication that the first sub-region is associated with the third workplace disruption score.

5. The method of claim 1, further comprising:
receiving, by the device, an indication of a selection of the first sub-region of the geographical area through a first user interface; and
causing presentation, by the device, of third interface data, wherein the third interface data is presented through a second user interface window that is separate from the first user interface, wherein the third interface data comprising additional metrics relating to the first sub-region, and wherein the first user interface and second user interface window are displayed concurrently.

6. The method of claim 1, further comprising:
receiving, by the device, a selection of a first filter associated with a third sub-region; and
causing presentation, by the device and based on the selection of the first filter, fourth interface data, the fourth interface data comprising a second visual map of the geographical area, the first sub-region, the second sub-region, and the third sub-region, wherein the second visual map includes a fourth visual indication that the third sub-region is associated with a fourth workplace disruption score.

7. The method of claim 1, further comprising:
determining, by the device, based on the first workplace disruption score, a first protocol indicative of a first workplace restriction associated with a physical location in the geographical area;
causing presentation, by the device, of third interface data, the third interface data indicative of the first workplace restriction;
determining, by the device, based on the second workplace disruption score, a second protocol indicative of a second workplace restriction associated with the physical location in the geographical area; and
causing presentation, by the device, of fourth interface data, the fourth interface data indicative of the second workplace restriction.

8. The method of claim 1, wherein the first visual indication includes the geographical area being presented as a first color, and wherein the second visual indication includes the geographical area being presented as a second color, the first color and second color being different.

9. A system comprising:
a processor; and
memory storing computer-executable instructions, that when executed by the processor, cause the processor to:
determine, by a device comprising at least one processor, a geographical area including a first sub-region and a second sub-region;
determine, by the device, a first metric associated with a disruptive event and the first sub-region and a second metric associated with the disruptive event and the second sub-region, the first metric and the second metric comprising a number of consecutive days that a severity of the event has decreased or increased;
determine, by the device and based on the first metric and the second metric, a first workplace disruption score associated with the disruptive event and the first sub-region and a second workplace disruption score associated with the disruptive event and the second sub-region at a first time; and
cause presentation, by the device, of first interface data, the first interface data comprising a visual map of the geographical area, the first sub-region, and the second sub-region, wherein the visual map includes a first visual indication that the first sub-region is associated with the first workplace disruption score and a second visual indication that the second sub-region is associated with the second workplace disruption score.

10. The system of claim 9, wherein the disruptive event is a pandemic event, and wherein the computer-executable instructions further cause the processor to:
predict, using a Bayesian structural equation and based on the first metric, a herd immunity date for the first sub-region.

11. The system of claim 9, wherein the computer-executable instructions further cause the processor to:
determine, by the device, a third workplace disruption score associated with the disruptive event and the first sub-region at a second time after the first time; and
cause presentation, by the device, of second interface data, the second interface data comprising the geographical area and a third visual indication that the first sub-region is associated with the third workplace disruption score.

12. The system of claim 9, wherein the computer-executable instructions further cause the processor to:
receive, by the device, an indication of a selection of the first sub-region of the geographical area through a first user interface; and
cause presentation, by the device, of third interface data, wherein the third interface data is presented through a second user interface window that is separate from the first user interface, wherein the third interface data comprising additional metrics relating to the first sub-region, and wherein the first user interface and second user interface window are displayed concurrently.

13. The system of claim 9, wherein the computer-executable instructions further cause the processor to:
receive, by the device, a selection of a first filter associated with a third sub-region; and
cause presentation, by the device and based on the selection of the first filter, fourth interface data, the fourth interface data comprising a second visual map of the geographical area, the first sub-region, the second sub-region, and the third sub-region, wherein the second visual map includes a fourth visual indication that the third sub-region is associated with a fourth workplace disruption score.

14. The system of claim 9, wherein the computer-executable instructions further cause the processor to:
determine, by the device, based on the first workplace disruption score, a first protocol indicative of a first workplace restriction associated with a physical location in the geographical area; and
cause presentation, by the device, of third interface data, the third interface data indicative of the first workplace restriction.

15. The system of claim 14, wherein the computer-executable instructions further cause the processor to:
determine, by the device, based on the second workplace disruption score, a second protocol indicative of a second workplace restriction associated with the physical location in the geographical area; and
cause presentation, by the device, of fourth interface data, the fourth interface data indicative of the second workplace restriction.

16. The system of claim 9, wherein the first visual indication includes the geographical area being presented as a first color, and wherein the second visual indication includes the geographical area being presented as a second color, the first color and second color being different.

17. A non-transitory computer-readable medium storing computer-executable instructions, that when executed by a processor, cause the processor to perform operations including:
determining, by a device comprising at least one processor, a geographical area including a first sub-region and a second sub-region;
determining, by the device, a first metric associated with a disruptive event and the first sub-region and a second metric associated with the disruptive event and the second sub-region, the first metric and the second metric comprising a number of consecutive days that a severity of the event has decreased or increased;
determining, by the device and based on the first metric and the second metric, a first workplace disruption score associated with the disruptive event and the first sub-region and a second workplace disruption score associated with the disruptive event and the second sub-region at a first time; and
causing presentation, by the device, of first interface data, the first interface data comprising a visual map of the geographical area, the first sub-region, and the second sub-region, wherein the visual map includes a first visual indication that the first sub-region is associated with the first workplace disruption score and a second visual indication that the second sub-region is associated with the second workplace disruption score.

18. The non-transitory computer-readable medium of claim 17, wherein the disruptive event is a pandemic event, and wherein the computer-executable instructions further cause the processor to perform operations including:
predicting, using a Bayesian structural equation and based on the first metric, a herd immunity date for the first sub-region.

19. The non-transitory computer-readable medium of claim 17, further comprising:
determining, by the device, a third workplace disruption score associated with the disruptive event and the first sub-region at a second time after the first time; and
causing presentation, by the device, of second interface data, the second interface data comprising the geographical area and a third visual indication that the first sub-region is associated with the third workplace disruption score.

20. The non-transitory computer-readable medium of claim 17, further comprising:
receiving, by the device, an indication of a selection of the first sub-region of the geographical area through the a first user interface; and
causing presentation, by the device, of third interface data, wherein the third interface data is presented through a second user interface window that is separate from the first user interface, wherein the third interface data comprising additional metrics relating to the first sub-region, and wherein the first user interface and second user interface window are displayed concurrently.
